SEQUENCE LISTING (5 sequences)

SEQ ID NO: 1 - protein, 305 aa, Homo sapiens
MAREDSVKCLRCLLYALNLLFWLMSISVLAVSAWMRDYLNNVLTLTAETRVEEAVILTYFPVVHPVMIAVCCFLIIVGMLGYCGTVKRNLLLLAWYFGSLLVIFCVELACGVWTYEQELMVPVQWSDMVTLKARMTNYGLPRYRWLTHAWNFFQREFKCCGVVYFTDWLEMTEMDWPPDSCCVREFPGCSKQAHQEDLSDLYQEGCGKKMYSFLRGTKQLQVLRFLGISIGVTQILAMILTITLLWALYYDRREPGTDQMMSLKNDNSQHLSCPSVELLKPSLSRIFEHTSMANSFNTHFEMEEL

SEQ ID NO: 2 - protein, 257 aa, Mus musculus
MAREDSVKCLRCLLYALNLLFWLMSISVLAVSAWMRDYLNNVLTLTAETRVEEAVILTYFPVVHPVMIAVCCFLIIVGMLGYCGTVKRNLLLLAWYFGTLLVIFCVELACGVWTYEQEVMVPVQWSDMVTLKARMTNYGLPRYRWLTHAWNYFQREGCGKKMYSFLRGTKQLQVLRFLGISIGVTQILAMILTITLLWALYYDRREPGTDQMLSLKNDTSQHLSCHSVELLKPSLSRIFEHTSMANSFNTHFEMEEL

SEQ ID NO: 3 - protein, 133 aa, Homo sapiens
MRKHVLAASFSMLSLLVIMGDTDSKTDSSFIMDSDPRRCMRHHYVDSISHPLYKCSSKMVLLARCEGHCSQASRSEPLVSFSTVLKQPFRSSCHCCRPQTSKLKALRLRCSGGMRLTATYRYILSCHCEECNS

SEQ ID NO: 4 - protein, 131 aa, Mus musculus
MRNHVLAASISMLSLLAIMGDTDSKTDSSFLMDSQRCMRHHYVDSISHPLYKCSSKMVLLARCEGHCSQASRSEPLVSFSTVLKQPFRSSCHCCRPQTSKLKALRLRCSGGMRLTATYRYILSCHCEECSS

SEQ ID NO: 5 - protein, 14 aa, Homo sapiens
CRREPGTDQMMSLK

(19) United States
(12) Patent Application Publication
(10) Pub. No.: US 2010/0129375 A1
(43) Pub. Date: May 27, 2010

(54) METHODS FOR INHIBITING OCULAR ANGIOGENESIS

(75) Inventors: Harald J. Junge, Brisbane, CA (US); Weilan Ye, Foster City, CA (US)

Correspondence Address: GENENTECH, INC., 1 DNA WAY, SOUTH SAN FRANCISCO, CA, 94080, US

(73) Assignee: Genentech, Inc., South San Francisco, CA (US)

(21) Appl. No.: 12/557,394

(22) Filed: Sep. 10, 2009

Related U.S. Application Data
(60) Provisional application No. 61/234,519, filed on Aug. 17, 2009.
(60) Provisional application No. 61/103,502, filed on Oct. 7, 2008.
(60) Provisional application No. 61/095,757, filed on Sep. 10, 2008.

(51) Int. Cl. A61K 39/395; A61K 38/00; A61P 27/02

(52) U.S. Cl. 424/145.1; 424/172.1; 424/158.1; 514/2

(57) ABSTRACT

The present invention provides methods of using TSPAN12 and Norrin antagonists to inhibit ocular vascular development and to treat related disorders.

### RELATED APPLICATIONS

This application claims benefit under 35 USC 119(e) to U.S. provisional application No. 61/095,757, filed Sep. 10, 2008; U.S. provisional application No. 61/103,502, filed Oct. 7, 2008; and U.S. provisional application No. 61/234,519, filed Aug. 17, 2009, the contents of each of which is incorporated herein by reference.

### FIELD OF THE INVENTION

The present invention relates generally to compositions and methods that are useful for treatment of conditions and diseases associated with angiogenesis. Specifically, the present invention relates to antagonists of tetraspanin 12 (TSPAN12) and Norrin.

### BACKGROUND OF THE INVENTION

It is now well established that angiogenesis is an important contributor to the pathogenesis of a variety of disorders. These include solid tumors and metastasis, intraocular neovascular diseases such as proliferative retinopathies, e.g., diabetic retinopathy, retinal vein occlusion (RVO), wet age-related macular degeneration (AMD), neovascular glaucoma, immune rejection of transplanted corneal tissue and other tissues, and rheumatoid arthritis. Duda et al. J. Clin. Oncology 25(26): 4033-42 (2007); Kesisis et al. Curr. Pharm. Des. 13: 2795-809 (2007); Zhang & Ma Prog. Ret. & Eye Res. 26: 1-37 (2007).

The retina receives its blood supply from retinal vessels, which supply the inner part of the retina, and choroidal vessels, which supply the outer part. Damage to retinal vessels occurs in several disease processes including diabetic retinopathy, retinopathy of prematurity, and central and branched retinal vein occlusions (ischemic retinopathies). Retinal ischemia from this damage results in undesirable neovascularization. Choroidal neovascularization occurs in a number of other disease processes, including AMD. In contrast, incomplete vascularization of the retina is a hallmark in patients with certain genetic diseases, e.g. familial exudative vitreoretinopathy (FEVR) and Norrie disease caused by mutation of the Wnt receptor Frizzled4 (Fzd4), the co-receptor LRP5 or the secreted ligand Norrin (Berger et al. Nature Genet. 1:199-203 (1992); Chen et al. Nature Genet. 1:204-208 (1992); Robitaille et al. Nature Genet. 32:326-30 (2002); Toomes et al. Am. J. Hum. Genet. 74:721-30 (2004)). Models for these genetic diseases are available in mice knocked out for the corresponding homologous genes.

Despite the many advances in the field of ocular angiogenesis, there remains a need to identify targets and develop means that can supplement or enhance the efficacy of existing therapies.

### SUMMARY OF THE INVENTION

The present invention is based, at least in part, on the discoveries that TSPAN12 is a component of the Norrin-induced, Frizzled-4- and LRP5-mediated signal transduction pathway and is required for the development of pathological angiogenesis. Therefore, Norrin and TSPAN12 are drug targets for inhibiting aberrant ocular angiogenesis, including conditions that do not harbor genetic mutations in the Norrin, TSPAN12, Frizzled-4 or LRP5 genes. Accordingly, the present invention provides novel methods for treating ocular diseases associated with angiogenesis using reagents that block Norrin or TSPAN12 activity.

In one aspect, the invention provides a method of reducing or inhibiting angiogenesis in a subject having an ocular disease or condition associated with angiogenesis, comprising administering to the subject a TSPAN12 antagonist. In some embodiments, the TSPAN12 antagonist is an anti-TSPAN12 antibody. In some embodiments, the TSPAN12 antagonist comprises a polypeptide fragment of TSPAN12, including an extracellular domain such as the second extracellular loop. In some embodiments, the antagonist further comprises an immunoglobulin constant region, e.g. an IgG Fc. In some embodiments, the ocular disease or condition is selected from the group consisting of: diabetic retinopathy, choroidal neovascularization (CNV), age-related macular degeneration (AMD), diabetic macular edema (DME), pathological myopia, von Hippel-Lindau disease, histoplasmosis of the eye, central retinal vein occlusion (CRVO), branched central retinal vein occlusion (BRVO), corneal neovascularization, retinal neovascularization, retinopathy of prematurity (ROP), subconjunctival hemorrhage, and hypertensive retinopathy.

In some embodiments, the method further comprises administering a second anti-angiogenic agent. In some embodiments, the second anti-angiogenic agent is administered prior to or subsequent to the administration of the TSPAN12 antagonist. In other embodiments, the second anti-angiogenic agent is administered concurrently with the TSPAN12 antagonist. In some embodiments, the second anti-angiogenic agent is an antagonist of Norrin or vascular endothelial cell growth factor (VEGF). In some embodiments, the Norrin antagonist or VEGF antagonist is an anti-Norrin antibody or an anti-VEGF antibody (e.g. ranibizumab).

In another aspect, the invention provides a method of reducing or inhibiting angiogenesis in a subject having an ocular disease or condition associated with angiogenesis, comprising administering to the subject a Norrin antagonist. In some embodiments, the Norrin antagonist is an anti-Norrin antibody. In some embodiments, the ocular disease or condition is selected from the group consisting of: diabetic retinopathy, CNV, AMD, DME, pathological myopia, von Hippel-Lindau disease, histoplasmosis of the eye, CRVO, BRVO, corneal neovascularization, retinal neovascularization, ROP, subconjunctival hemorrhage, and hypertensive retinopathy.

In some embodiments, the method further comprises administering a second anti-angiogenic agent. In some embodiments, the second anti-angiogenic agent is administered prior to or subsequent to the administration of the Norrin antagonist. In other embodiments, the second anti-angiogenic agent is administered concurrently with the Norrin antagonist. In some embodiments, the second anti-angiogenic agent is an antagonist of VEGF, e.g. an anti-VEGF antibody such as ranibizumab.

In another aspect, the invention provides a method of treating an ocular disease or condition associated with undesired angiogenesis in a subject comprising administering to the subject a TSPAN12 antagonist. In some embodiments, the TSPAN12 antagonist is an anti-TSPAN12 antibody. In some embodiments, the TSPAN12 antagonist comprises a polypeptide fragment of TSPAN12, including an extracellular domain such as the second extracellular loop. In some embodiments, the antagonist further comprises an immunoglobulin constant region, e.g. an IgG Fc. In some embodiments, the ocular disease or condition is selected from the group consisting of: proliferative retinopathies including proliferative diabetic retinopathy, CNV, AMD, diabetic and other ischemia-related retinopathies, DME, pathological myopia, von Hippel-Lindau disease, histoplasmosis of the eye, CRVO, BRVO, corneal neovascularization, retinal neovascularization, ROP, subconjunctival hemorrhage, and hypertensive retinopathy.

In some embodiments, the method further comprises administering a second anti-angiogenic agent. In some embodiments, the second anti-angiogenic agent is administered prior to or subsequent to the administration of the TSPAN12 antagonist. In other embodiments, the second anti-angiogenic agent is administered concurrently with the TSPAN12 antagonist. In some embodiments, the second anti-angiogenic agent is an antagonist of Norrin or VEGF. In some embodiments, the Norrin antagonist or VEGF antagonist is an anti-Norrin antibody or an anti-VEGF antibody (e.g. ranibizumab).

In another aspect, the invention provides a method of treating an ocular disease or condition associated with undesired angiogenesis in a subject comprising administering administering to the subject a Norrin antagonist. In some embodiments, the Norrin antagonist is an anti-Norrin antibody. In some embodiments, the ocular disease or condition is selected from the group consisting of: proliferative retinopathies including proliferative diabetic retinopathy, CNV, AMD, diabetic and other ischemia-related retinopathies, DME, pathological myopia, von Hippel-Lindau disease, histoplasmosis of the eye, CRVO, BRVO, corneal neovascularization, retinal neovascularization, ROP, subconjunctival hemorrhage, and hypertensive retinopathy.

In some embodiments, the method further comprises administering a second anti-angiogenic agent. In some embodiments, the second anti-angiogenic agent is administered prior to or subsequent to the administration of the Norrin antagonist. In other embodiments, the second anti-angiogenic agent is administered concurrently with the Norrin antagonist. In some embodiments, the second anti-angiogenic agent is an antagonist of VEGF, e.g. an anti-VEGF antibody such as ranibizumab.

In another aspect, the invention provides a method of producing an antibody using a peptide consisting essentially of amino acids CRREPGTDQMMSLK (SEQ ID NO: 5). In some embodiments, the method comprises immunizing an animal with the peptide. In some embodiments, the method comprises screening a library (e.g. a Fab library) to identify an antibody or antibody fragment that binds the peptide. In some embodiments, the invention provides antibodies generated by any such method. In some embodiments, the invention provides a method of detecting TSPAN12 using any such antibodies.

In another aspect, the invention provides in vitro and in vivo methods of inhibiting FZD4 multimer formation comprising administering a TSPAN12 antagonist. In another aspect, the invention provides in vitro and in vivo methods of inhibiting Norrin-mediated signaling comprising administering a TSPAN12 antagonist. In some embodiments, the TSPAN12 antagonist is an anti-TSPAN12 antibody. In some embodiments, the TSPAN12 antagonist comprises a polypeptide fragment of TSPAN12, including an extracellular domain such as the second extracellular loop. In some embodiments, the antagonist further comprises an immunoglobulin constant region, e.g. an IgG Fc.

In another aspect, the invention provides a method of treating a subject with a congenital ocular disease caused by a genetic mutation in any of the Norrin, TSPAN12, FZD4 or LRP5 genes comprising administering to the subject an agent that enhances FZD4 multimer formation. In some embodiments, the disease is FEVR, Norrie disease or Coate's disease. In some embodiments, the agent that increases FZD4 multimer formation is selected from the group consisting of: Norrin, anti-FZD4 antibody, anti-LRP5 antibody, and a bispecific anti-FZD4/anti-LRP5 antibody. In some embodiments, the genetic mutation impairs FZD4-mediated signaling. In some embodiments, the genetic mutation in the subject produces an aberrant protein product in the subject selected from the group consisting of: Norrin-C95R, FZD4-M105V and FZD4-M157V. In some embodiments, the presence of the mutation in the subject is detected prior to treating the subject.

### BRIEF DESCRIPTION OF THE DRAWINGS

FIG. 1 shows representative Retinopathy of Prematurity (ROP) retinal sections for wildtype (top) and TSPAN12 knockout (KO) mice (bottom).

FIG. 2 shows quantitative results of the number of neovascular nuclei observed in ROP retinal sections from wildtype (WT; left) and TSPAN12 KO mice (Hom; right).

FIG. 3 shows that TSPAN12 enhances Norrin-mediated signal transduction by Fzd4/LRP5.

FIG. 4 shows that the TSPAN12 enhancement of Norrin-mediated signal transduction is specific to Fzd4.

FIG. 5 shows that TSPAN12 does not enhance Wnt3a-mediated signal transduction.

FIG. 6 shows that Norrin binds to Fzd4 but not to LRP5 or TSPAN12.

FIG. 7 shows that Norrin bound to cells expressing Fzd4 but not cells expressing Fzd5, LRP5 or TSPAN12 and that Norrin does not coimmunoprecipitate with TSPAN12.

FIG. 8 shows that TSPAN12 does not associate with LRP5.

FIG. 9 shows that TSPAN12 does not enhance Norrin binding to Fzd4.

FIG. 10 shows that coexpression of TSPAN12 does not alter expression of Fzd4 on the plasma membrane.

FIG. 11 shows the higher order structures formed by wildtype Norrin and C95R mutant Norrin.

FIG. 12 shows that overexpression of TSPAN12 can compensate for the defects in monomeric C95R Norrin.

FIG. 13 shows that TSPAN12 regulates FZD4 clustering during signalling.

### DETAILED DESCRIPTION OF THE PREFERRED EMBODIMENTS

### Definitions

Unless defined otherwise, technical and scientific terms used herein have the same meaning as commonly understood by one of ordinary skill in the art to which this invention belongs. See, e.g. Singleton et al., Dictionary of Microbiology and Molecular Biology 2nd ed., J. Wiley & Sons (New York, N.Y. 1994); Sambrook et al., Molecular Cloning, A Laboratory Manual, Cold Spring Harbor Press (Cold Spring Harbor, N.Y. 1989). For purposes of the present invention, certain terms are defined below.

As used herein, the terms “TSPAN12,” “TSPAN12 polypeptide,” “Norrin,” and “Norrin polypeptide” refer to a polypeptide having the amino acid sequence of a TSPAN12 or Norrin polypeptide derived from nature, regardless of its mode of preparation or species. Thus, such polypeptides can have the amino acid sequence of naturally occurring TSPAN12 or Norrin from a human, a mouse, or any other species. A full-length human TSPAN12 amino acid sequence is:

(SEQ ID NO: 1)

MAREDSVKCLRCLLYALNLLFWLMSISVLAVSAWMRDYLNNVLTLTAETR
VEEAVILTYFPVVHPVMIAVCCFLIIVGMLGYCGTVKRNLLLLAWYFGSL
LVIFCVELACGVWTYEQELMVPVQWSDMVTLKARMTNYGLPRYRWLTHAW
NFFQREFKCCGVVYFTDWLEMTEMDWPPDSCCVREFPGCSKQAHQEDLSD
LYQEGCGKKMYSFLRGTKQLQVLRFLGISIGVTQILAMILTITLLWALYY
DRREPGTDQMMSLKNDNSQHLSCPSVELLKPSLSRIFEHTSMANSFNTHF
EMEEL.

A full-length mouse TSPAN12 amino acid sequence is:

(SEQ ID NO: 2)

MAREDSVKCLRCLLYALNLLFWLMSISVLAVSAWMRDYLNNVLTLTAETR
VEEAVILTYFPVVHPVMIAVCCFLIIVGMLGYCGTVKRNLLLLAWYFGTL
LVIFCVELACGVWTYEQEVMVPVQWSDMVTLKARMTNYGLPRYRWLTHAW
NYFQREGCGKKMYSFLRGTKQLQVLRFLGISIGVTQILAMILTITLLWAL
YYDRREPGTDQMLSLKNDTSQHLSCHSVELLKPSLSRIFEHTSMANSFNT
HFEMEEL.

A full-length human Norrin amino acid sequence is:

(SEQ ID NO: 3)

MRKHVLAASFSMLSLLVIMGDTDSKTDSSFIMDSDPRRCMRHHYVDSISH
PLYKCSSKMVLLARCEGHCSQASRSEPLVSFSTVLKQPFRSSCHCCRPQT
SKLKALRLRCSGGMRLTATYRYILSCHCEECNS.

A full-length mouse Norrin amino acid sequence is:

(SEQ ID NO: 4)

MRNHVLAASISMLSLLAIMGDTDSKTDSSFLMDSQRCMRHHYVDSISHPL
YKCSSKMVLLARCEGHCSQASRSEPLVSFSTVLKQPFRSSCHCCRPQTSK
LKALRLRCSGGMRLTATYRYILSCHCEECSS.

Such TSPAN12 or Norrin polypeptides can be isolated from nature or can be produced by recombinant and/or synthetic means.

“Isolated” in reference to a polypeptide means that it has been purified from an natural source or has been prepared by recombinant or synthetic methods and purified. A “purified” polypeptide is substantially free of other polypeptides or peptides. “Substantially free” here means less than about 5%, preferably less than about 2%, more preferably less than about 1%, even more preferably less than about 0.5%, most preferably less than about 0.1% contamination with other source proteins.

The term “antagonist” is used in the broadest sense, and includes any molecule that partially or fully blocks, inhibits, or neutralizes a biological activity of a polypeptide. For example, an antagonist of TSPAN12 or Norrin would partially or fully block, inhibit, or neutralize the ability of TSPAN12 or Norrin to transduce or initiate Norrin-induced signaling or to enable pathologic vessel formation in the eye. Suitable antagonist molecules specifically include antagonist antibodies or antibody fragments, fragments or amino acid sequence variants of a native TSPAN12 or Norrin polypeptide, peptides, soluble fragments of TSPAN12 or Norrin co-receptor(s), antisense RNAs, ribozymes, RNAi, small organic molecules, etc. Methods for identifying antagonists of a TSPAN12 or Norrin polypeptide may comprise contacting the TSPAN12 or Norrin polypeptide with a candidate antagonist molecule and measuring a detectable change in one or more biological activities normally associated with the polypeptide.

“Active” or “activity” for the purposes herein refers to form(s) of TSPAN12 or Norrin which retain a biological and/or an immunological activity, wherein “biological” activity refers to a biological function caused by TSPAN12 or Norrin other than the ability to induce the production of an antibody and an “immunological” activity refers to the ability to induce the production of an antibody against an antigenic epitope possessed by TSPAN12 or Norrin. Principal biological activities of TSPAN12 and Norrin are transduction or initiation of Norrin-induced signaling and inducing pathologic vessel formation in the eye.

“TSPAN12 co-receptor” or “Norrin co-receptor” refer to molecules to which TSPAN12 or Norrin binds and which mediate a biological activity of TSPAN12 or Norrin.

The term “antibody” herein is used in the broadest sense and specifically covers human, non-human (e.g. murine) and humanized monoclonal antibodies (including full length monoclonal antibodies), polyclonal antibodies, multispecific antibodies (e.g., bispecific antibodies), and antibody fragments so long as they exhibit the desired biological activity.

“Native antibodies” are usually heterotetrameric glycoproteins of about 150,000 daltons, composed of two identical light (L) chains and two identical heavy (H) chains. Each light chain is linked to a heavy chain by one covalent disulfide bond, while the number of disulfide linkages varies among the heavy chains of different immunoglobulin isotypes. Each heavy and light chain also has regularly spaced intra-chain disulfide bridges. Each heavy chain has at one end a variable domain (V_{H}) followed by a number of constant domains. Each light chain has a variable domain at one end (V_{L}) and a constant domain at its other end; the constant domain of the light chain is aligned with the first constant domain of the heavy chain, and the light-chain variable domain is aligned with the variable domain of the heavy chain. Particular amino acid residues are believed to form an interface between the light- and heavy-chain variable domains.

Papain digestion of antibodies produces two identical antigen-binding fragments, called “Fab” fragments, each with a single antigen-binding site, and a residual “Fc” fragment, whose name reflects its ability to crystallize readily. Pepsin treatment yields an F(ab′)₂ fragment that has two antigen-combining sites and is still capable of cross-linking antigen.

“Fv” is the minimum antibody fragment that contains a complete antigen recognition and binding site. This region consists of a dimer of one heavy chain and one light chain variable domain in tight, non-covalent association.

The Fab fragment also contains the constant domain of the light chain and the first constant domain (CH1) of the heavy chain. Fab′ fragments differ from Fab fragments by the addition of a few residues at the carboxyl terminus of the heavy chain CH1 domain including one or more cysteine(s) from the antibody hinge region. Fab′-SH is the designation herein for Fab′ in which the cysteine residue(s) of the constant domains bear a free thiol group. F(ab′)₂ antibody fragments originally were produced as pairs of Fab′ fragments which have hinge cysteines between them. Other chemical couplings of antibody fragments are also known.

The “light chains” of antibodies (immunoglobulins) from any vertebrate species can be assigned to one of two clearly distinct types, called kappa (κ) and lambda (λ) based on the amino acid sequences of their constant domains.

Depending on the amino acid sequence of the constant domain of their heavy chains, immunoglobulins can be assigned to different classes. There are five major classes of immunoglobulins: IgA, IgD, IgE, IgG, and IgM, and several of these may be further divided into subclasses (isotypes), e.g., IgG1, IgG2, IgG3, IgG4, IgA1, and IgA2. The heavy-chain constant domains that correspond to the different classes of immunoglobulins are called α, δ, ε, γ, and μ, respectively. The subunit structures and three-dimensional configurations of different classes of immunoglobulins are well known.

“Antibody fragments” comprise a portion of a full-length antibody, generally the antigen binding or variable domain thereof. Examples of antibody fragments include Fab, Fab′, F(ab′)₂, and Fv fragments.

The term “monoclonal antibody” as used herein refers to an antibody obtained from a population of substantially homogeneous antibodies, i.e., the individual antibodies comprising the population are identical except for possible naturally occurring mutations that may be present in minor amounts. Monoclonal antibodies are highly specific, being directed against a single antigenic site. Furthermore, in contrast to conventional (polyclonal) antibody preparations that typically include different antibodies directed against different determinants (epitopes), each monoclonal antibody is directed against a single determinant on the antigen. The modifier “monoclonal” indicates the character of the antibody as being obtained from a substantially homogeneous population of antibodies, and is not to be construed as requiring production of the antibody by any particular method. For example, the monoclonal antibodies to be used in accordance with the present invention may be made by the hybridoma method first described by Kohler et al., Nature 256:495 (1975), or may be made by recombinant DNA methods (see, e.g., U.S. Pat. No. 4,816,567). The “monoclonal antibodies” may also be isolated from phage antibody libraries using the techniques described in Clackson et al., Nature 352:624-628 (1991) and Marks et al., J. Mol. Biol. 222:581-597 (1991), for example.

The monoclonal antibodies herein specifically include “chimeric” antibodies in which a portion of the heavy and/or light chain is identical with or homologous to corresponding sequences in antibodies derived from a particular species or belonging to a particular antibody class or subclass, while the remainder of the chain(s) is identical with or homologous to corresponding sequences in antibodies derived from another species or belonging to another antibody class or subclass, as well as fragments of such antibodies, so long as they exhibit the desired biological activity (U.S. Pat. No. 4,816,567; and Morrison et al., Proc. Natl. Acad. Sci. USA 81:6851-6855 (1984)).

“Humanized” forms of non-human (e.g., murine) antibodies are chimeric antibodies that contain minimal sequence derived from non-human immunoglobulin. For the most part, humanized antibodies are human immunoglobulins (recipient antibody) in which hypervariable region residues of the recipient are replaced by hypervariable region residues from a non-human species (donor antibody) such as mouse, rat, rabbit or non-human primate having the desired specificity, affinity, and capacity. In some instances, framework region (FR) residues of the human immunoglobulin are replaced by corresponding non-human residues. Furthermore, humanized antibodies may comprise residues that are not found in the recipient antibody or in the donor antibody. These modifications are made to further refine antibody performance. In general, the humanized antibody will comprise substantially all of at least one, and typically two, variable domains, in which all or substantially all of the hypervariable regions correspond to those of a non-human immunoglobulin and all or substantially all of the FRs are those of a human immunoglobulin sequence. The humanized antibody optionally also will comprise at least a portion of an immunoglobulin constant region (Fc), typically that of a human immunoglobulin. For further details, see Jones et al. Nature 321:522-525 (1986); Reichmann et al. Nature 332:323-329 (1988); and Presta Curr. Op. Struct. Biol. 2:593-596 (1992).

As used herein, “treatment” is an approach for obtaining beneficial or desired clinical results. For purposes of this invention, beneficial or desired clinical results include, but are not limited to, alleviation of symptoms, diminishment of extent of disease, stabilized (i.e., not worsening) state of disease, delay or slowing of disease progression, amelioration or palliation of the disease state, and remission (whether partial or total), whether detectable or undetectable. “Treatment” is an intervention performed with the intention of preventing the development or altering the pathology of a disorder. Accordingly, “treatment” may refer to therapeutic treatment or prophylactic or preventative measures. Those in need of treatment include those already with the disorder as well as those in which the disorder is to be prevented. Specifically, the treatment may directly prevent, slow down or otherwise decrease the pathology of cellular degeneration or damage, such as the pathology of tumor cells in cancer treatment, or may render the cells more susceptible to treatment by other therapeutic agents.

“Chronic” administration refers to administration of the agent(s) in a continuous mode as opposed to an acute mode, so as to maintain the initial therapeutic effect (activity) for an extended period of time. “Intermittent” administration is treatment that is not consecutively done without interruption, but rather is cyclic in nature.

An “intraocular neovascular disease” is a disease characterized by ocular neovascularization. Examples of intraocular neovascular diseases include, but are not limited to, proliferative retinopathies including proliferative diabetic retinopathy, choroidal neovascularization (CNV), age-related macular degeneration (AMD), diabetic and other ischemia-related retinopathies, diabetic macular edema (DME), pathological myopia, von Hippel-Lindau disease, histoplasmosis of the eye, central retinal vein occlusion (CRVO), branched central retinal vein occlusion (BRVO), corneal neovascularization, retinal neovascularization, retinopathy of prematurity (ROP), subconjunctival hemorrhage, and hypertensive retinopathy. Preferably, an intraocular neovascular disease excludes conditions that result from genetic mutations in any of the Norrin, TSPAN12, Frizzled-4 or LRP5 genes. For example, an intraocular neovascular disease of the invention preferably excludes FEVR and Norrie disease.

The “pathology” of a disease includes all phenomena that compromise the well-being of the patient.

Administration “in combination with” one or more further therapeutic agents includes simultaneous (concurrent) and consecutive administration in any order.

“Carriers” as used herein include pharmaceutically acceptable carriers, excipients, or stabilizers which are nontoxic to the cell or mammal being exposed thereto at the dosages and concentrations employed. Often the physiologically acceptable carrier is an aqueous pH buffered solution. Examples of physiologically acceptable carriers include buffers such as phosphate, citrate, and other organic acids; antioxidants including ascorbic acid; low molecular weight (less than about 10 residues) polypeptide; proteins, such as serum albumin, gelatin, or immunoglobulins; hydrophilic polymers such as polyvinylpyrrolidone; amino acids such as glycine, glutamine, asparagine, arginine or lysine; monosaccharides, disaccharides, and other carbohydrates including glucose, mannose, or dextrins; chelating agents such as EDTA; sugar alcohols such as mannitol or sorbitol; salt-forming counterions such as sodium; and/or nonionic surfactants such as TWEEN™, polyethylene glycol (PEG), and PLURONICS™.

A “small molecule” is defined herein to have a molecular weight below about 500 Daltons.

### METHODS FOR CARRYING OUT THE INVENTION

Preparation and Identification of Antagonists of TSPAN12 or Norrin Activity

Screening assays for antagonist drug candidates are designed to identify compounds that bind or complex with TSPAN12 or Norrin polypeptides, or otherwise interfere with their activity and/or interaction with other cellular proteins.

Small molecules may have the ability to act as TSPAN12 or Norrin antagonists and thus to be therapeutically useful. Such small molecules may include naturally occurring small molecules, synthetic organic or inorganic compounds and peptides. However, small molecules in the present invention are not limited to these forms. Extensive libraries of small molecules are commercially available and a wide variety of assays are taught herein or are well known in the art to screen these molecules for the desired activity.

In some embodiments, small molecule TSPAN12 or Norrin antagonists are identified by their ability to inhibit one or more of the biological activities of TSPAN12 or Norrin. Thus a candidate compound is contacted with TSPAN12 or Norrin and a biological activity of TSPAN12 or Norrin is then assessed. In one embodiment the ability of TSPAN12 or Norrin to transduce or initiate Norrin-mediated signaling is assessed. A compound is identified as an antagonist where the biological activity of TSPAN12 or Norrin is inhibited.

Compounds identified as TSPAN12 or Norrin antagonists may be used in the methods of the present invention. For example, TSPAN12 or Norrin antagonists may be used to treat intraocular neovascular disease.

A variety of well-known animal models (including, e.g., models of retinopathy of prematurity and laser-induced choroidal neovascularization; Ruiz-Edera & Verkman Invest. Opthalmol. & Vis. Sci. 48(10): 4802-10 (2007), Yu et al. Invest. Opthalmol. & Vis. Sci. 49(6): 2599-605 (2007)) can be used to further understand the role of TSPAN12 or Norrin in the development and pathogenesis of intraocular neovascular disease, and to test the efficacy of candidate therapeutic agents, including antibodies and other antagonists of native TSPAN12 or Norrin polypeptides, such as small-molecule antagonists. The in vivo nature of such models makes them particularly predictive of responses in human patients.

### Antibody Binding Studies

The ability of antibodies to bind to and inhibit the effect of TSPAN12 or Norrin on Wnt signaling reporter cells is tested. Exemplary methods are provided in Example 2, but other methods will be readily apparent to one of ordinary skill in the art. Exemplary antibodies include polyclonal, monoclonal, humanized, bispecific, and heteroconjugate antibodies, the preparation of which is described herein.

Antibody binding studies may be carried out in any known assay method, such as competitive binding assays, direct and indirect sandwich assays, and immunoprecipitation assays. Zola, Monoclonal Antibodies: A Manual of Techniques (CRC Press, Inc., 1987), pp. 147-158.

Competitive binding assays rely on the ability of a labeled standard to compete with the test sample analyte for binding with a limited amount of antibody. The amount of target protein in the test sample is inversely proportional to the amount of standard that becomes bound to the antibodies. To facilitate determining the amount of standard that becomes bound, the antibodies preferably are insolubilized before or after the competition, so that the standard and analyte that are bound to the antibodies may conveniently be separated from the standard and analyte that remain unbound.

Sandwich assays involve the use of two antibodies, each capable of binding to a different immunogenic portion, or epitope, of the protein to be detected. In a sandwich assay, the test sample analyte is bound by a first antibody that is immobilized on a solid support, and thereafter a second antibody binds to the analyte, thus forming an insoluble three-part complex. See, e.g., U.S. Pat. No. 4,376,110. The second antibody may itself be labeled with a detectable moiety (direct sandwich assays) or may be measured using an anti-immunoglobulin antibody that is labeled with a detectable moiety (indirect sandwich assay). For example, one type of sandwich assay is an ELISA assay, in which case the detectable moiety is an enzyme.

For immunohistochemistry, the tissue sample may be fresh or frozen or may be embedded in paraffin and fixed with a preservative such as formalin, for example.

The compositions useful in the treatment of cardiovascular, endothelial, and angiogenic disorders include, without limitation, antibodies, small organic and inorganic molecules, peptides, phosphopeptides, antisense, siRNA and ribozyme molecules, triple-helix molecules, etc., that inhibit the expression and/or activity of the target gene product.

More specific examples of potential antagonists include an polypeptide that binds to TSPAN12 or Norrin, and, in particular, antibodies including, without limitation, poly- and monoclonal antibodies and antibody fragments, single-chain antibodies, anti-idiotypic antibodies, and chimeric or humanized versions of such antibodies or fragments, as well as human antibodies and antibody fragments. For TSPAN12, certain extracellular domain(s) may also serve as antagonists (see, e.g., Ho et al. J. Virol. 80(13): 6487-96 (2006); Hemler Nature Rev. Drug Discovery 7: 747-58 (2008)). Alternatively, a potential antagonist may be a closely related protein, for example, a mutated form of TSPAN12 or Norrin that interacts with co-receptor(s) but imparts no effect, thereby competitively inhibiting the action of TSPAN12 or Norrin.

Another potential TSPAN12 or Norrin antagonist is an antisense RNA or DNA construct prepared using antisense technology, where, e.g., an antisense RNA or DNA molecule acts to block directly the translation of mRNA by hybridizing to targeted mRNA and preventing protein translation. Antisense technology can be used to control gene expression through triple-helix formation or antisense DNA or RNA, both of which methods are based on binding of a polynucleotide to DNA or RNA. For example, the 5′ coding portion of the polynucleotide sequence, which encodes the mature TSPAN12 and Norrin polypeptides herein, is used to design an antisense RNA oligonucleotide of from about 10 to 40 base pairs in length. A DNA oligonucleotide is designed to be complementary to a region of the gene involved in transcription (triple helix—see, Lee et al., Nucl. Acids Res. 6:3073 (1979); Cooney et al., Science 241:456 (1988); Dervan et al., Science 251:1360 (1991)), thereby preventing transcription and the production of TSPAN12 or Norrin. A sequence “complementary” to a portion of an RNA, as referred to herein, means a sequence having sufficient complementarity to be able to hybridize with the RNA, forming a stable duplex; in the case of double-stranded antisense nucleic acids, a single strand of the duplex DNA may thus be tested, or triplex helix formation may be assayed.

The ability to hybridize will depend on both the degree of complementarity and the length of the antisense nucleic acid. Generally, the longer the hybridizing nucleic acid, the more base mismatches with an RNA it may contain and still form a stable duplex (or triplex, as the case may be). One skilled in the art can ascertain a tolerable degree of mismatch by use of standard procedures to determine the melting point of the hybridized complex. The antisense RNA oligonucleotide hybridizes to the mRNA in vivo and blocks translation of the mRNA molecule into TSPAN12 (antisense—Okano, Neurochem. 56:560 (1991); Oligodeoxynucleotides as Antisense Inhibitors of Gene Expression (CRC Press: Boca Raton, Fla., 1988).

The antisense oligonucleotides can be DNA or RNA or chimeric mixtures or derivatives or modified versions thereof, single-stranded or double-stranded. The oligonucleotide can be modified at the base moiety, sugar moiety, or phosphate backbone, for example, to improve stability of the molecule, hybridization, etc. The oligonucleotide may include other appended groups such as peptides (e.g., for targeting host cell receptors in vivo), or agents facilitating transport across the cell membrane (see, e.g., Letsinger, et al., Proc. Natl. Acad. Sci. U.S.A. 86:6553-6556 (1989); Lemaitre, et al., Proc. Natl. Acad. Sci. U.S.A. 84:648-652 (1987); PCT Publication No. WO88/09810, published Dec. 15, 1988) or the blood-brain barrier (see, e.g., PCT Publication No. WO89/10134, published Apr. 25, 1988), hybridization-triggered cleavage agents (see, e.g., Krol et al., BioTechniques 6:958-976 (1988)) or intercalating agents (see, e.g., Zon, Pharm. Res. 5:539-549 (1988)). To this end, the oligonucleotide may be conjugated to another molecule, e.g., a peptide, hybridization triggered cross-linking agent, transport agent, hybridization-triggered cleavage agent, etc.

The antisense oligonucleotide may comprise at least one modified base moiety which is selected from the group including but not limited to 5-fluorouracil, 5-bromouracil, 5-chlorouracil, 5-iodouracil, hypoxanthine, xanthine, 4-acetylcytosine, 5-(carboxyhydroxylmethyl) uracil, 5-carboxymethylaminomethyl-2-thiouridine, 5-carboxymethylaminomethyluracil, dihydrouracil, beta-D-galactosylqueosine, inosine, N6-isopentenyladenine, 1-methylguanine, 1-methylinosine, 2,2-dimethylguanine, 2-methyladenine, 2-methylguanine, 3-methylcytosine, 5-methylcytosine, N6-adenine, 7-methylguanine, 5-methylaminomethyluracil, 5-methoxyaminomethyl-2-thiouracil, beta-D-mannosylqueosine, 5′-methoxycarboxymethyluracil, 5-methoxyuracil, 2-methylthio-N-6-isopentenyladenine, uracil-5-oxyacetic acid (v), wybutoxosine, pseudouracil, queosine, 2-thiocytosine, 5-methyl-2-thiouracil, 2-thiouracil, 4-thiouracil, 5-methyluracil, uracil-5-oxyacetic acid methylester, uracil-5-oxyacetic acid (v), 5-methyl-2-thiouracil, 3-(3-amino-3-N2-carboxypropyl) uracil, (acp3)w, and 2,6-diaminopurine.

The antisense oligonucleotide may also comprise at least one modified sugar moiety selected from the group including but not limited to arabinose, 2-fluoroarabinose, xylulose, and hexose.

In yet another embodiment, the antisense oligonucleotide comprises at least one modified phosphate backbone selected from the group consisting of a phosphorothioate, a phosphorodithioate, a phosphoramidothioate, a phosphoramidate, a phosphordiamidate, a methylphosphonate, an alkyl phosphotriester, and a formacetal or analog thereof.

In yet another embodiment, the antisense oligonucleotide is an anomeric oligonucleotide. An anomeric oligonucleotide forms specific double-stranded hybrids with complementary RNA in which, contrary to the usual units, the strands run parallel to each other (Gautier, et al., Nucl. Acids Res. 15:6625-6641 (1987)). The oligonucleotide is a 2′-0-methylribonucleotide (Inoue, et al., Nucl. Acids Res. 15:6131-6148 (1987)), or a chimeric RNA-DNA analogue (Inoue, et al., FEBS Lett. 215:327-330 (1987)).

In some embodiments, the antagonists are inhibitory duplex RNAs, e.g. siRNA, shRNA, etc.

Oligonucleotides of the invention may be synthesized by standard methods known in the art, e.g., by use of an automated DNA synthesizer (such as are commercially available from Biosearch, Applied Biosystems, etc.). As examples, phosphorothioate oligonucleotides may be synthesized by the method of Stein, et al. (Nucl. Acids Res. 16:3209 (1988)), methylphosphonate oligonucleotides can be prepared by use of controlled pore glass polymer supports (Sarin, et al., Proc. Natl. Acad. Sci. U.S.A. 85:7448-7451 (1988)), etc.

The oligonucleotides described above can also be delivered to cells such that the antisense RNA or DNA may be expressed in vivo to inhibit production of TSPAN12 or Norrin. When antisense DNA is used, oligodeoxyribonucleotides derived from the translation-initiation site, e.g., between about −10 and +10 positions of the target gene nucleotide sequence, are preferred.

Potential antagonists further include small molecules that bind to TSPAN12 or Norrin, thereby blocking its activity. Examples of small molecules include, but are not limited to, small peptides or peptide-like molecules, preferably soluble peptides, and synthetic non-peptidyl organic or inorganic compounds.

Additional potential antagonists are ribozymes, which are enzymatic RNA molecules capable of catalyzing the specific cleavage of RNA. Ribozymes act by sequence-specific hybridization to the complementary target RNA, followed by endonucleolytic cleavage. Specific ribozyme cleavage sites within a potential RNA target can be identified by known techniques. For further details see, e.g., Rossi, Current Biology 4:469-471 (1994), and PCT publication No. WO 97/33551 (published Sep. 18, 1997).

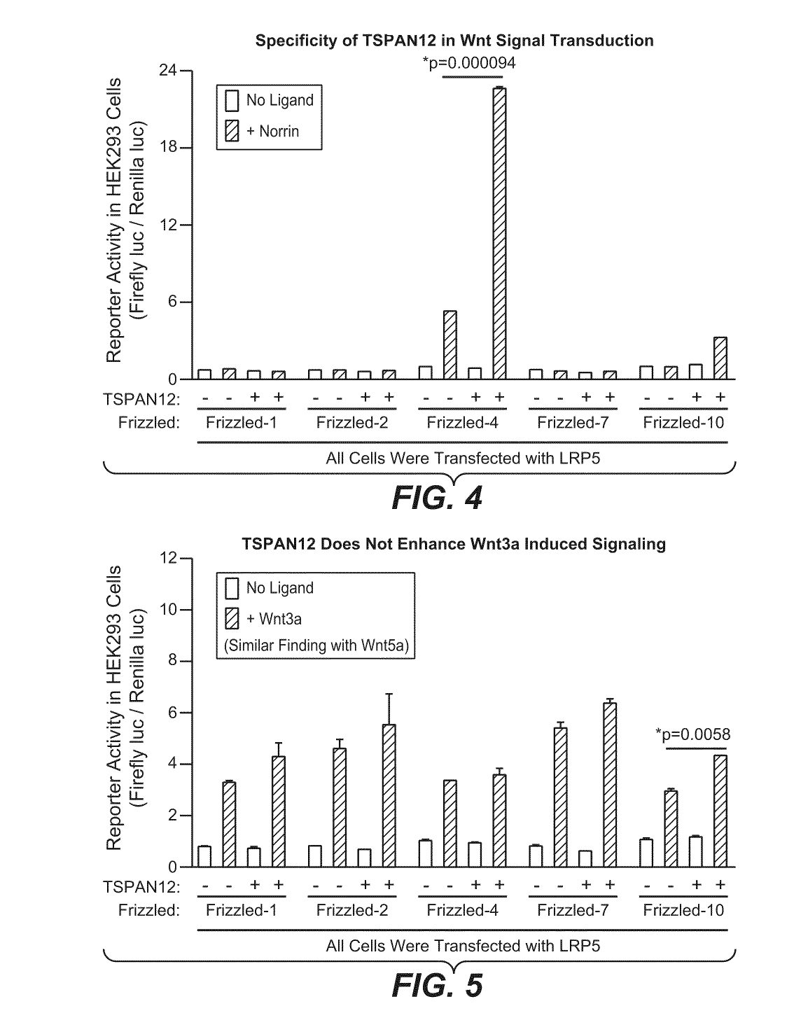

While ribozymes that cleave mRNA at site specific recognition sequences can be used to destroy target gene mRNAs, the use of hammerhead ribozymes is preferred. Hammerhead ribozymes cleave mRNAs at locations dictated by flanking regions which form complementary base pairs with the target mRNA. The sole requirement is that the target mRNA have the following sequence of two bases: 5′-UG-3′. The construction and production of hammerhead ribozymes is well known in the art and is described more fully in Myers, Molecular Biology and Biotechnology: A Comprehensive Desk Reference, VCH Publishers, New York (1995), (see especially FIG. 4, page 833) and in Haseloff and Gerlach, Nature, 334:585-591 (1988), which is incorporated herein by reference in its entirety.

Preferably the ribozyme is engineered so that the cleavage recognition site is located near the 5′ end of the target gene mRNA, i.e., to increase efficiency and minimize the intracellular accumulation of non-functional mRNA transcripts.

The ribozymes of the present invention also include RNA endoribonucleases (hereinafter “Cech-type ribozymes”) such as the one which occurs naturally in Tetrahymena thermophila (known as the IVS, or L-19 IVS RNA) and which has been extensively described by Thomas Cech and collaborators (Zaug, et al., Science, 224:574-578 (1984); Zaug and Cech, Science, 231:470-475 (1986); Zaug, et al., Nature, 324:429-433 (1986); published International patent application No. WO 88/04300 by University Patents Inc.; Been and Cech, Cell, 47:207-216 (1986)). The Cech-type ribozymes have an eight base pair active site that hybridizes to a target RNA sequence whereafter cleavage of the target RNA takes place. The invention encompasses those Cech-type ribozymes that target eight base-pair active site sequences that are present in the target gene.

As in the antisense approach, the ribozymes can be composed of modified oligonucleotides (e.g., for improved stability, targeting, etc.) and should be delivered to cells that express the target gene in vivo. A preferred method of delivery involves using a DNA construct “encoding” the ribozyme under the control of a strong constitutive pol III or pol II promoter, so that transfected cells will produce sufficient quantities of the ribozyme to destroy endogenous target gene messages and inhibit translation. Because ribozymes, unlike antisense molecules, are catalytic, a lower intracellular concentration is required for efficiency.

Nucleic acid molecules in triple-helix formation used to inhibit transcription should be single-stranded and composed of deoxynucleotides. The base composition of these oligonucleotides is designed such that it promotes triple-helix formation via Hoogsteen base-pairing rules, which generally require sizeable stretches of purines or pyrimidines on one strand of a duplex. For further details see, e.g., PCT publication No. WO 97/33551, supra.

The TSPAN12 or Norrin antagonists can also be useful in treating intraocular diseases including, but are not limited to, proliferative retinopathies including proliferative diabetic retinopathy, choroidal neovascularization (CNV), age-related macular degeneration (AMD), diabetic and other ischemia-related retinopathies, diabetic macular edema (DME), pathological myopia, von Hippel-Lindau disease, histoplasmosis of the eye, central retinal vein occlusion (CRVO), branched central retinal vein occlusion (BRVO), corneal neovascularization, retinal neovascularization, retinopathy of prematurity (ROP), subconjunctival hemorrhage, and hypertensive retinopathy.

### Administration Protocols, Schedules, Doses, and Formulations

The TSPAN12 or Norrin antagonists are pharmaceutically useful as a prophylactic and therapeutic agent for various disorders and diseases as set forth above.

Therapeutic compositions of the antagonists are prepared for storage by mixing the desired molecule having the appropriate degree of purity with optional pharmaceutically acceptable carriers, excipients, or stabilizers (Remington's Pharmaceutical Sciences, 16th edition, Osol, A. ed. (1980)), in the form of lyophilized formulations or aqueous solutions. Acceptable carriers, excipients, or stabilizers are nontoxic to recipients at the dosages and concentrations employed, and include buffers such as phosphate, citrate, and other organic acids; antioxidants including ascorbic acid and methionine; preservatives (such as octadecyldimethylbenzyl ammonium chloride; hexamethonium chloride; benzalkonium chloride, benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g., Zn-protein complexes); and/or non-ionic surfactants such as TWEEN™, PLURONICS™ or polyethylene glycol (PEG).

Additional examples of such carriers include ion exchangers, alumina, aluminum stearate, lecithin, serum proteins, such as human serum albumin, buffer substances such as phosphates, glycine, sorbic acid, potassium sorbate, partial glyceride mixtures of saturated vegetable fatty acids, water, salts, or electrolytes such as protamine sulfate, disodium hydrogen phosphate, potassium hydrogen phosphate, sodium chloride, zinc salts, colloidal silica, magnesium trisilicate, polyvinyl pyrrolidone, cellulose-based substances, and polyethylene glycol. Carriers for topical or gel-based forms of antagonist include polysaccharides such as sodium carboxymethylcellulose or methylcellulose, polyvinylpyrrolidone, polyacrylates, polyoxyethylene-polyoxypropylene-block polymers, polyethylene glycol, and wood wax alcohols. For all administrations, conventional depot forms are suitably used. Such forms include, for example, microcapsules, nano-capsules, liposomes, plasters, inhalation forms, nose sprays, sublingual tablets, and sustained-release preparations. TSPAN12 or Norrin antagonists will typically be formulated in such vehicles at a concentration of about 0.1 mg/ml to 100 mg/ml.

Another formulation comprises incorporating TSPAN12 or Norrin antagonist into formed articles. Such articles can be used in modulating endothelial cell growth and angiogenesis. In addition, tumor invasion and metastasis may be modulated with these articles.

TSPAN12 or Norrin antagonists to be used for in vivo administration must be sterile. This is readily accomplished by filtration through sterile filtration membranes, prior to or following lyophilization and reconstitution. If in lyophilized form, TSPAN12 or Norrin antagonist is typically formulated in combination with other ingredients for reconstitution with an appropriate diluent at the time for use. An example of a liquid formulation of TSPAN12 or Norrin antagonist is a sterile, clear, colorless unpreserved solution filled in a single-dose vial for subcutaneous injection. Preserved pharmaceutical compositions suitable for repeated use may contain, for example, depending mainly on the indication and type of polypeptide:

TSPAN12 or Norrin antagonist;

a buffer capable of maintaining the pH in a range of maximum stability of the polypeptide or other molecule in solution, preferably about 4-8;

a detergent/surfactant primarily to stabilize the polypeptide or molecule against agitation-induced aggregation;

an isotonifier;

a preservative selected from the group of phenol, benzyl alcohol and a benzethonium halide, e.g., chloride; and

water.

If the detergent employed is non-ionic, it may, for example, be polysorbates (e.g., POLYSORBATE™ (TWEEN™) 20, 80, etc.) or poloxamers (e.g., POLOXAMER™ 188). The use of non-ionic surfactants permits the formulation to be exposed to shear surface stresses without causing denaturation of the polypeptide. Further, such surfactant-containing formulations may be employed in aerosol devices such as those used in a pulmonary dosing, and needleless jet injector guns (see, e.g., EP 257,956).

An isotonifier may be present to ensure isotonicity of a liquid composition of TSPAN12 or Norrin antagonist, and includes polyhydric sugar alcohols, preferably trihydric or higher sugar alcohols, such as glycerin, erythritol, arabitol, xylitol, sorbitol, and mannitol. These sugar alcohols can be used alone or in combination. Alternatively, sodium chloride or other appropriate inorganic salts may be used to render the solutions isotonic.

The buffer may, for example, be an acetate, citrate, succinate, or phosphate buffer depending on the pH desired. The pH of one type of liquid formulation of this invention is buffered in the range of about 4 to 8, preferably about physiological pH.

The preservatives phenol, benzyl alcohol and benzethonium halides, e.g., chloride, are known antimicrobial agents that may be employed.

Therapeutic polypeptide compositions described herein generally are placed into a container having a sterile access port, for example, an intravenous solution bag or vial having a stopper pierceable by a hypodermic injection needle. The formulations may be administered as repeated intravenous (i.v.), subcutaneous (s.c.), or intramuscular (i.m.) injections, or as aerosol formulations suitable for intranasal or intrapulmonary delivery (for intrapulmonary delivery see, e.g., EP 257,956). The formulations are preferably administered as intravitreal (IVT) or subconjuctival delivery.

Therapeutic polypeptides can also be administered in the form of sustained-released preparations. Suitable examples of sustained-release preparations include semipermeable matrices of solid hydrophobic polymers containing the protein, which matrices are in the form of shaped articles, e.g., films, or microcapsules. Examples of sustained-release matrices include polyesters, hydrogels (e.g., poly(2-hydroxyethyl-methacrylate) as described by Langer et al., J. Biomed. Mater. Res. 15:167-277 (1981) and Langer, Chem. Tech. 12:98-105 (1982) or poly(vinylalcohol)), polylactides (U.S. Pat. No. 3,773,919, EP 58,481), copolymers of L-glutamic acid and gamma ethyl-L-glutamate (Sidman et al., Biopolymers 22:547-556 (1983)), non-degradable ethylene-vinyl acetate (Langer et al., supra), degradable lactic acid-glycolic acid copolymers such as the Lupron Depot® (injectable microspheres composed of lactic acid-glycolic acid copolymer and leuprolide acetate), and poly-D-(−)-3-hydroxybutyric acid (EP 133,988).

While polymers such as ethylene-vinyl acetate and lactic acid-glycolic acid enable release of molecules for over 100 days, certain hydrogels release proteins for shorter time periods. When encapsulated proteins remain in the body for a long time, they may denature or aggregate as a result of exposure to moisture at 37° C., resulting in a loss of biological activity and possible changes in immunogenicity. Rational strategies can be devised for protein stabilization depending on the mechanism involved. For example, if the aggregation mechanism is discovered to be intermolecular S—S bond formation through thio-disulfide interchange, stabilization may be achieved by modifying sulfhydryl residues, lyophilizing from acidic solutions, controlling moisture content, using appropriate additives, and developing specific polymer matrix compositions.

Sustained-release TSPAN12 or Norrin antagonist compositions also include liposomally entrapped antagonists. Such liposomes are prepared by methods known per se: DE 3,218,121; Epstein et al., Proc. Natl. Acad. Sci. USA 82:3688-3692 (1985); Hwang et al., Proc. Natl. Acad. Sci. USA 77:4030-4034 (1980); EP 52,322; EP 36,676; EP 88,046; EP 143,949; EP 142,641; Japanese patent application 83-118008; U.S. Pat. Nos. 4,485,045 and 4,544,545; and EP 102,324. Ordinarily the liposomes are of the small (about 200-800 Angstroms) unilamellar type in which the lipid content is greater than about 30 mol. % cholesterol, the selected proportion being adjusted for the optimal therapy.

The therapeutically effective dose of TSPAN12 or Norrin antagonist will, of course, vary depending on such factors as the pathological condition to be treated (including prevention), the method of administration, the type of compound being used for treatment, any co-therapy involved, the patient's age, weight, general medical condition, medical history, etc., and its determination is well within the skill of a practicing physician. Accordingly, it will be necessary for the therapist to titer the dosage and modify the route of administration as required to obtain the maximal therapeutic effect.

With the above guidelines, the effective dose generally is within the range of from about 0.001 to about 1.0 mg/kg, more preferably about 0.01-1.0 mg/kg, most preferably about 0.01-0.1 mg/kg.

The route of TSPAN12 or Norrin antagonist administration is in accord with known methods, e.g., by injection or infusion by intravenous, intramuscular, intracerebral, intraperitoneal, intracerobrospinal, subcutaneous, intraocular (including intravitreal), intraarticular, intrasynovial, intrathecal, oral, topical, or inhalation routes, or by sustained-release systems as noted.

If a peptide or small molecule is employed as an antagonist, it is preferably administered orally or non-orally in the form of a liquid or solid to mammals.

Examples of pharmacologically acceptable salts of molecules that form salts and are useful hereunder include alkali metal salts (e.g., sodium salt, potassium salt), alkaline earth metal salts (e.g., calcium salt, magnesium salt), ammonium salts, organic base salts (e.g., pyridine salt, triethylamine salt), inorganic acid salts (e.g., hydrochloride, sulfate, nitrate), and salts of organic acid (e.g., acetate, oxalate, p-toluenesulfonate).

### Combination Therapies

The effectiveness of TSPAN12 or Norrin antagonists in preventing or treating the disorder in question may be improved by administering the active agent serially or in combination with another agent that is effective for those purposes, either in the same composition or as separate compositions.

For example, TSPAN12 or Norrin antagonists used to treat angiogenesis associated conditions such as ocular diseases may be combined with other agents. In particular, it is desirable to use TSPAN12 or Norrin antagonists in combination with each other or with another anti-angiogenic agent. In some embodiments, the TSPAN12 or Norrin antagonist is used in combination with a VEGF antagonist, e.g. an antibody, e.g. ranibizumab.

The effective amounts of the therapeutic agents administered in combination with TSPAN12 or Norrin antagonist will be at the physician's or veterinarian's discretion. Dosage administration and adjustment is done to achieve maximal management of the conditions to be treated. The dose will additionally depend on such factors as the type of the therapeutic agent to be used and the specific patient being treated. Typically, the amount employed will be the same dose as that used, if the given therapeutic agent is administered without TSPAN12 or Norrin.

### TSPAN12 or Norrin Antibodies

Some of the most promising drug candidates according to the present invention are antibodies and antibody fragments that may inhibit the production of TSPAN12 or Norrin and/or reduce an activity of TSPAN12 or Norrin.

### Polyclonal Antibodies

Methods of preparing polyclonal antibodies are known to the skilled artisan. Polyclonal antibodies can be raised in a mammal, for example, by one or more injections of an immunizing agent and, if desired, an adjuvant. Typically, the immunizing agent and/or adjuvant will be injected in the mammal by multiple subcutaneous or intraperitoneal injections. The immunizing agent may include the TSPAN12 or Norrin polypeptide or a fusion protein thereof. It may be useful to conjugate the immunizing agent to a protein known to be immunogenic in the mammal being immunized Examples of such immunogenic proteins include, but are not limited to, keyhole limpet hemocyanin, serum albumin, bovine thyroglobulin, and soybean trypsin inhibitor. Examples of adjuvants that may be employed include Freund's complete adjuvant and MPL-TDM adjuvant (monophosphoryl Lipid A or synthetic trehalose dicorynomycolate). The immunization protocol may be selected by one skilled in the art without undue experimentation.

### Monoclonal Antibodies

The anti-TSPAN12 or anti-Norrin antibodies may, alternatively, be monoclonal antibodies. Monoclonal antibodies may be prepared using hybridoma methods, such as those described by Kohler and Milstein, Nature 256:495 (1975). In a hybridoma method, a mouse, hamster, or other appropriate host animal is typically immunized with an immunizing agent to elicit lymphocytes that produce or are capable of producing antibodies that will specifically bind to the immunizing agent. Alternatively, the lymphocytes may be immunized in vitro.

The immunizing agent will typically include the TSPAN12 or Norrin polypeptide or a fusion protein thereof. Generally, either peripheral blood lymphocytes (“PBLs”) are used if cells of human origin are desired, or spleen cells or lymph node cells are used if non-human mammalian sources are desired. The lymphocytes are then fused with an immortalized cell line using a suitable fusing agent, such as polyethylene glycol, to form a hybridoma cell. Goding, Monoclonal Antibodies: Principles and Practice (New York: Academic Press, 1986), pp. 59-103. Immortalized cell lines are usually transformed mammalian cells, particularly myeloma cells of rodent, bovine, and human origin. Usually, rat or mouse myeloma cell lines are employed. The hybridoma cells may be cultured in a suitable culture medium that preferably contains one or more substances that inhibit the growth or survival of the unfused, immortalized cells. For example, if the parental cells lack the enzyme hypoxanthine guanine phosphoribosyl transferase (HGPRT or HPRT), the culture medium for the hybridomas typically will include hypoxanthine, aminopterin, and thymidine (“HAT medium”), which substances prevent the growth of HGPRT-deficient cells.

Preferred immortalized cell lines are those that fuse efficiently, support stable high-level expression of antibody by the selected antibody-producing cells, and are sensitive to a medium such as HAT medium. More preferred immortalized cell lines are murine myeloma lines, which can be obtained, for instance, from the Salk Institute Cell Distribution Center, San Diego, Calif. and the American Type Culture Collection, Manassas, Va. Human myeloma and mouse-human heteromyeloma cell lines also have been described for the production of human monoclonal antibodies. Kozbor, J. Immunol. 133:3001 (1984); Brodeur et al., Monoclonal Antibody Production Techniques and Applications (Marcel Dekker, Inc.: New York, 1987) pp. 51-63.

The culture medium in which the hybridoma cells are cultured can then be assayed for the presence of monoclonal antibodies directed against the TSPAN12 or Norrin polypeptide. Preferably, the binding specificity of monoclonal antibodies produced by the hybridoma cells is determined by immunoprecipitation or by an in vitro binding assay, such as radioimmunoassay (RIA) or enzyme-linked immunoabsorbent assay (ELISA). Such techniques and assays are known in the art. The binding affinity of the monoclonal antibody can, for example, be determined by the Scatchard analysis of Munson and Pollard, Anal. Biochem. 107:220 (1980).

After the desired hybridoma cells are identified, the clones may be subcloned by limiting dilution procedures and grown by standard methods. Goding, supra. Suitable culture media for this purpose include, for example, Dulbecco's Modified Eagle's Medium and RPMI-1640 medium. Alternatively, the hybridoma cells may be grown in vivo as ascites in a mammal.

The monoclonal antibodies secreted by the subclones may be isolated or purified from the culture medium or ascites fluid by conventional immunoglobulin purification procedures such as, for example, protein A-Sepharose, hydroxylapatite chromatography, gel electrophoresis, dialysis, or affinity chromatography.

The monoclonal antibodies may also be made by recombinant DNA methods, such as those described in U.S. Pat. No. 4,816,567. DNA encoding the monoclonal antibodies of the invention can be readily isolated and sequenced using conventional procedures (e.g., by using oligonucleotide probes that are capable of binding specifically to genes encoding the heavy and light chains of murine antibodies). The hybridoma cells of the invention serve as a preferred source of such DNA. Once isolated, the DNA may be placed into expression vectors, which are then transfected into host cells such as simian COS cells, Chinese hamster ovary (CHO) cells, or myeloma cells that do not otherwise produce immunoglobulin protein, to obtain the synthesis of monoclonal antibodies in the recombinant host cells. The DNA also may be modified, for example, by substituting the coding sequence for human heavy- and light-chain constant domains in place of the homologous murine sequences (U.S. Pat. No. 4,816,567; Morrison et al., supra) or by covalently joining to the immunoglobulin coding sequence all or part of the coding sequence for a non-immunoglobulin polypeptide. Such a non-immunoglobulin polypeptide can be substituted for the constant domains of an antibody of the invention, or can be substituted for the variable domains of one antigen-combining site of an antibody of the invention to create a chimeric bivalent antibody.

The antibodies may be monovalent antibodies. Methods for preparing monovalent antibodies are well known in the art. For example, one method involves recombinant expression of immunoglobulin light chain and modified heavy chain. The heavy chain is truncated generally at any point in the Fc region so as to prevent heavy-chain crosslinking. Alternatively, the relevant cysteine residues are substituted with another amino acid residue or are deleted so as to prevent crosslinking.

In vitro methods are also suitable for preparing monovalent antibodies. Digestion of antibodies to produce fragments thereof, particularly Fab fragments, can be accomplished using routine techniques known in the art.

Human and Humanized Antibodies

The anti-TSPAN12 or anti-Norrin antibodies may further comprise humanized antibodies or human antibodies. Humanized forms of non-human (e.g., murine) antibodies are chimeric immunoglobulins, immunoglobulin chains, or fragments thereof (such as Fv, Fab, Fab′, F(ab′)₂, or other antigen-binding subsequences of antibodies) that contain minimal sequence derived from non-human immunoglobulin. Humanized antibodies include human immunoglobulins (recipient antibody) in which residues from a CDR of the recipient are replaced by residues from a CDR of a non-human species (donor antibody) such as mouse, rat, or rabbit having the desired specificity, affinity, and capacity. In some instances, Fv framework residues of the human immunoglobulin are replaced by corresponding non-human residues. Humanized antibodies may also comprise residues that are found neither in the recipient antibody nor in the imported CDR or framework sequences. In general, the humanized antibody will comprise substantially all of at least one, and typically two, variable domains, in which all or substantially all of the CDR regions correspond to those of a non-human immunoglobulin, and all or substantially all of the FR regions are those of a human immunoglobulin consensus sequence. The humanized antibody preferably also will comprise at least a portion of an immunoglobulin constant region (Fc), typically that of a human immunoglobulin. Jones et al., Nature 321:522-525 (1986); Riechmann et al., Nature 332:323-329 (1988); Presta, Curr. Op. Struct. Biol. 2:593-596 (1992).

Methods for humanizing non-human antibodies are well known in the art. Generally, a humanized antibody has one or more amino acid residues introduced into it from a source that is non-human. These non-human amino acid residues are often referred to as “import” residues, which are typically taken from an “import” variable domain. Humanization can be essentially performed following the method of Winter and co-workers (Jones et al., Nature 321:522-525 (1986); Riechmann et al., Nature 332:323-327 (1988); Verhoeyen et al., Science 239:1534-1536 (1988)), by substituting rodent CDRs or CDR sequences for the corresponding sequences of a human antibody. Accordingly, such “humanized” antibodies are chimeric antibodies (U.S. Pat. No. 4,816,567), wherein substantially less than an intact human variable domain has been substituted by the corresponding sequence from a non-human species. In practice, humanized antibodies are typically human antibodies in which some CDR residues and possibly some FR residues are substituted by residues from analogous sites in rodent antibodies.

Human antibodies can also be produced using various techniques known in the art, including phage display libraries. Hoogenboom and Winter, J. Mol. Biol. 227:381 (1991); Marks et al., J. Mol. Biol. 222:581 (1991). The techniques of Cole et al. and Boerner et al. are also available for the preparation of human monoclonal antibodies. Cole et al., Monoclonal Antibodies and Cancer Therapy, Alan R. Liss, p. 77 (1985) and Boerner et al., J. Immunol. 147(1):86-95 (1991). Similarly, human antibodies can be made by introducing human immunoglobulin loci into transgenic animals, e.g., mice in which the endogenous immunoglobulin genes have been partially or completely inactivated. Upon challenge, human antibody production is observed that closely resembles that seen in humans in all respects, including gene rearrangement, assembly, and antibody repertoire. This approach is described, for example, in U.S. Pat. Nos. 5,545,807; 5,545,806; 5,569,825; 5,625,126; 5,633,425; and 5,661,016, and in the following scientific publications: Marks et al., Bio/Technology 10:779-783 (1992); Lonberg et al., Nature 368:856-859 (1994); Morrison, Nature, 368:812-813 (1994); Fishwild et al., Nature Biotechnology 14:845-851 (1996); Neuberger, Nature Biotechnology 14:826 (1996); Lonberg and Huszar, Intern. Rev. Immunol. 13:65-93 (1995).

Bispecific Antibodies

Bispecific antibodies are monoclonal, preferably human or humanized, antibodies that have binding specificities for at least two different antigens. In the present case, one of the binding specificities is for the TSPAN12 or Norrin polypeptide, the other one is for the polypeptide or any other antigen. Examples include a cell-surface protein or receptor or receptor subunit.

Methods for making bispecific antibodies are known in the art. Traditionally, the recombinant production of bispecific antibodies is based on the co-expression of two immunoglobulin heavy-chain/light-chain pairs, where the two heavy chains have different specificities. Milstein & Cuello, Nature 305:537-539 (1983). Because of the random assortment of immunoglobulin heavy and light chains, these hybridomas (quadromas) produce a potential mixture of ten different antibody molecules, of which only one has the correct bispecific structure. The purification of the correct molecule is usually accomplished by affinity chromatography steps. Similar procedures are disclosed in WO 93/08829, published 13 May 1993, and in Traunecker et al., EMBO J. 10:3655-3659 (1991).

Antibody variable domains with the desired binding specificities (antibody-antigen combining sites) can be fused to immunoglobulin constant-domain sequences. The fusion preferably is with an immunoglobulin heavy-chain constant domain, comprising at least part of the hinge, CH2, and CH3 regions. It is preferred to have the first heavy-chain constant region (CH1) containing the site necessary for light-chain binding present in at least one of the fusions. DNAs encoding the immunoglobulin heavy-chain fusions and, if desired, the immunoglobulin light chain, are inserted into separate expression vectors, and are co-transfected into a suitable host organism. For further details of generating bispecific antibodies, see, for example, Suresh et al., Methods in Enzymology 121:210 (1986).

Heteroconjugate Antibodies

Heteroconjugate antibodies are composed of two covalently joined antibodies. Such antibodies have, for example, been proposed to target immune-system cells to unwanted cells (U.S. Pat. No. 4,676,980), and for treatment of HIV infection. WO 91/00360; WO 92/200373; EP 03089. It is contemplated that the antibodies may be prepared in vitro using known methods in synthetic protein chemistry, including those involving crosslinking agents. For example, immunotoxins may be constructed using a disulfide-exchange reaction or by forming a thioether bond. Examples of suitable reagents for this purpose include iminothiolate and methyl-4-mercaptobutyrimidate and those disclosed, for example, in U.S. Pat. No. 4,676,980.

Immunoliposomes

The antibodies disclosed herein may also be formulated as immunoliposomes. Liposomes containing the antibody are prepared by methods known in the art, such as described in Epstein et al., Proc. Natl. Acad. Sci. USA 82:3688 (1985); Hwang et al., Proc. Natl. Acad. Sci. USA 77:4030 (1980); and U.S. Pat. Nos. 4,485,045 and 4,544,545. Liposomes with enhanced circulation time are disclosed in U.S. Pat. No. 5,013,556.

Particularly useful liposomes can be generated by the reverse-phase evaporation method with a lipid composition comprising phosphatidylcholine, cholesterol, and PEG-derivatized phosphatidylethanolamine (PEG-PE). Liposomes are extruded through filters of defined pore size to yield liposomes with the desired diameter. Fab′ fragments of the antibody of the present invention can be conjugated to the liposomes as described in Martin et al., J. Biol. Chem. 257:286-288 (1982) via a disulfide-interchange reaction. A chemotherapeutic agent (such as Doxorubicin) is optionally contained within the liposome. See, Gabizon et al., J. National Cancer Inst. 81(19):1484 (1989).

Pharmaceutical Compositions of Antibodies

Antibodies specifically binding an TSPAN12 or Norrin polypeptide identified herein, as well as other molecules identified by the screening assays disclosed herein, can be administered for the treatment of various disorders as noted above and below in the form of pharmaceutical compositions.

The formulation herein may also contain more than one active compound as necessary for the particular indication being treated, preferably those with complementary activities that do not adversely affect each other. Alternatively, or in addition, the composition may comprise an agent that enhances its function. Such molecules are suitably present in combination in amounts that are effective for the purpose intended.

The active ingredients may also be entrapped in microcapsules prepared, for example, by coacervation techniques or by interfacial polymerization, for example, hydroxymethylcellulose or gelatin-microcapsules and poly-(methylmethacylate) microcapsules, respectively, in colloidal drug delivery systems (for example, liposomes, albumin microspheres, microemulsions, nano-particles, and nanocapsules) or in macroemulsions. Such techniques are disclosed in Remington's Pharmaceutical Sciences, supra.

The formulations to be used for in vivo administration must be sterile. This is readily accomplished by filtration through sterile filtration membranes.

Sustained-release preparations may be prepared. Suitable examples of sustained-release preparations include semipermeable matrices of solid hydrophobic polymers containing the antibody, which matrices are in the form of shaped articles, e.g., films, or microcapsules. Examples of sustained-release matrices include polyesters, hydrogels (for example, poly(2-hydroxyethyl-methacrylate), or poly(vinylalcohol)), polylactides (U.S. Pat. No. 3,773,919), copolymers of L-glutamic acid and γ ethyl-L-glutamate, non-degradable ethylene-vinyl acetate, degradable lactic acid-glycolic acid copolymers such as the LUPRON DEPOT® (injectable microspheres composed of lactic acid-glycolic acid copolymer and leuprolide acetate), and poly-D-(−)-3-hydroxybutyric acid. While polymers such as ethylene-vinyl acetate and lactic acid-glycolic acid enable release of molecules for over 100 days, certain hydrogels release proteins for shorter time periods. When encapsulated antibodies remain in the body for a long time, they may denature or aggregate as a result of exposure to moisture at 37° C., resulting in a loss of biological activity and possible changes in immunogenicity. Rational strategies can be devised for stabilization depending on the mechanism involved. For example, if the aggregation mechanism is discovered to be intermolecular S—S bond formation through thio-disulfide interchange, stabilization may be achieved by modifying sulfhydryl residues, lyophilizing from acidic solutions, controlling moisture content, using appropriate additives, and developing specific polymer matrix compositions.

Methods of Treatment using the Antibody

It is contemplated that the antibodies to TSPAN12 or Norrin may be used to treat various angiogenesis associated conditions as noted above.

The antibodies are administered to a mammal, preferably a human, in accord with known methods, such as intravenous administration as a bolus or by continuous infusion over a period of time, by intravitreal, intramuscular, intraperitoneal, intracerobrospinal, subcutaneous, intra-articular, intrasynovial, intrathecal, oral, topical, or inhalation routes. Intravitreal administration of the antibody is preferred.

In one embodiment, pathological ocular neovascularization is attacked in combination therapy. The anti-TSPAN12 and/or anti-Norrin antibody and another antibody (e.g., anti-VEGF) are administered to patients at therapeutically effective doses.

For example, depending on the type and severity of the disorder, about 1 μg/kg to 50 mg/kg (e.g., 0.1-20 mg/kg) of antibody is an initial candidate dosage for administration to the patient, whether, for example, by one or more separate administrations, or by continuous infusion. A typical daily or weekly dosage might range from about 1 μg/kg to 100 mg/kg or more, depending on the factors mentioned above. For repeated administrations over several days or longer, depending on the condition, the treatment is repeated or sustained until a desired suppression of disorder symptoms occurs. However, other dosage regimens may be useful. The progress of this therapy is easily monitored by conventional techniques and assays, including, for example, radiographic tumor imaging.

The following Examples are offered for illustrative purposes only, and are not intended to limit the scope of the present invention in any way.

The disclosures of all patent and literature references cited in the present specification are hereby incorporated by reference in their entirety.

### EXAMPLES

Commercially available reagents referred to in the Examples were used according to manufacturer's instructions unless otherwise indicated. All references cited herein are hereby incorporated by reference.

### Example 1

### TSPAN12 is Involved in Normal and Pathological Angiogenesis

Although the coding region for TSPAN12 is known in several organisms, including humans and mice (see, e.g., GenBank® Accession Nos. NM_{—}012338 for hTSPAN12 and NM_{—}173007 for mTSPAN12), no function for it has been identified. To begin to elucidate its function, TSPAN12 knockout (KO) mice were generated using conventional methods. Specifically, the targeting construct was electroporated into 129/SvEvBrd (Lex-2) ES cells and targeted clones were identified using a southern blot assay. Cells from a targeted clone were injected into C57BL/6 (albino) blastocysts. The resulting chimeras were mated to C57BL/6 (albino) females to generate mice that were heterozygous for the mutation and subsequently backcrossed onto a C57/BL6 background (>N3) and used for phenotypic analysis and other experiments. TSPAN12−/− mice were viable and fertile.

We generated a rabbit polyclonal serum named αTSPAN12-Anaspec-C against the c-terminal intracellular peptide CRREPGTDQMMSLK (SEQ ID NO: 5) and affinity purified it. We also raised a second polyclonal serum named α-TSPAN12-Josman-B against a his-tagged TSPAN12 fragment corresponding to amino acids 116-221 (aa116-221-His6) expressed in bacteria and purified. The targeting in the mutant mice was confirmed by Southern blot, PCR and western blot from lysates of P1 heads, which were enriched for TSPAN12 by immunoprecipitation using αTSPAN12-Anaspec-C.

We first analyzed expression of TSPAN12 in various tissues and found that it was expressed in the developing retinal vasculature at P15 and in the meninges at P1. Little or no expression was detected in non-CNS vasculature. However, we observed no large-scale significant morphological alteration of the NFL vasculature in isolectin-stained retinal wholemount preparations from TSPAN12 KO mice that were processed according to published procedures (Gerhardt et al., J. Cell. Biol. 161(6):1163-77 (2003)). Accordingly, we analyze the TSPAN12 mutant mice for phenotypes in a variety of ocular disease models.

Mouse pups from eight TSPAN12 (C57BL/6) het×het crosses were generated and used in a mouse model of retinopathy of prematurity (ROP). Six litters, along with their nursing mothers, were placed in 75% oxygen (hyperoxia) for a period of five days beginning at P7. At P12, the animals were returned to room air condition (normoxia) and maintained for another five days (P17). Tail biopsies were used for genotyping and the 37 animals broke down as follows: homozygous wildtype n=8, heterozygous n=21, homozygous mutant n=8.

On P17, the right-eye was placed in 4% PFA and the left-eye was placed in Davidson's fixative. The cornea and lens were removed from left eyes for paraffin processing and sectioning. Eye cups were placed in the block with the iris side down, facing the sectioning surface. Sections spaced 16 microns apart were obtained and analyzed for neovascularization using the Aperio ScanScope® equipped with a custom designed nuclear algorithm. Briefly, neovascular tufts intimately associated with the nerve fiber layer (NFL) were identified as regions of interest for the algorithm quantification. A minimum of 30 sections per case, containing neovascular nuclei were quantified for assessment of retinal neovascularization.

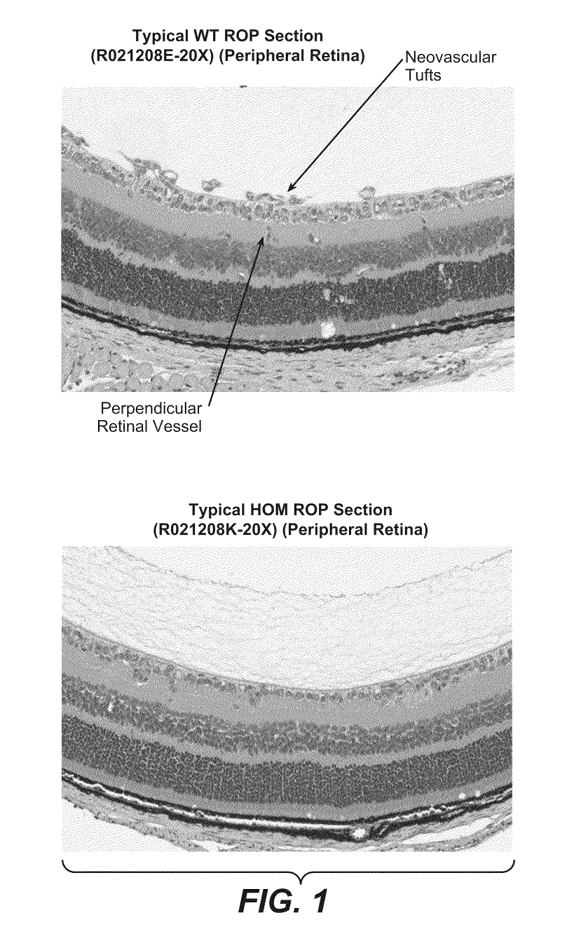

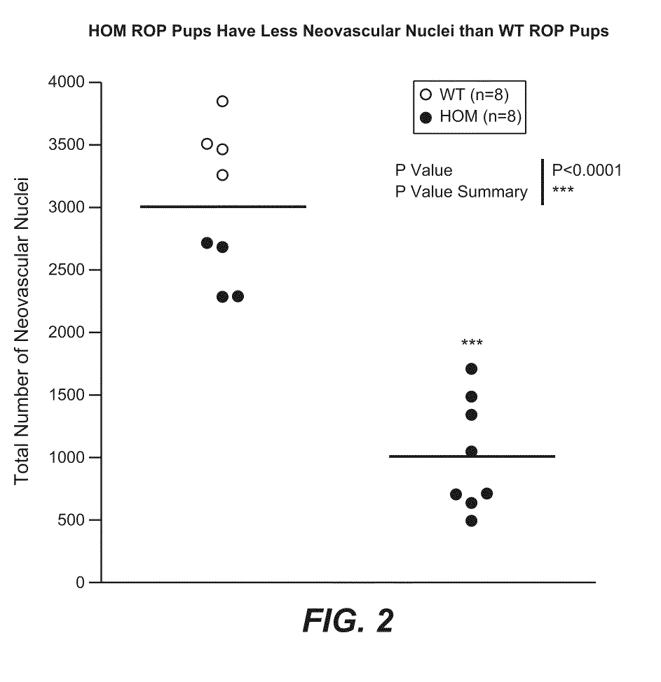

In wildtype animals raised under conditions designed to model ROP, neovascular nuclei at the retinal/vitreal interface were identified (FIG. 1). In contrast, homozygous TSPAN12 mutant mice had markedly fewer neovascular nuclei (FIG. 2). These data indicated that TSPAN12 is required for pathologic neovascularization in this ROP model.

After observing this phenotype in the ROP model, we analyzed development of the retinal vasculature on a finer scale. In the murine retina a superficial vascular plexus in the NFL is established from the optic nerve head through a combination of sprouting, migration and remodeling between postnatal days P0-P10. Subsequently, vessels sprout into the outer plexiform layer (OPL) and into the inner plexiform layer (IPL), where two capillary beds are established, resulting in a three-layered vascular architecture. By P8 we found the development of the NFL vasculature was not significantly changed in retinal wholemounts of TSPAN12−/− mice. In cross-sections we found that the formation of capillaries in the OPL had begun by P11 in the TSPAN12+/+ mice while sprouting was completely absent in TSPAN12−/− mice. The fact that TSPAN12 expression was detected in the vasculature but not other retinal tissues together with the observation that the retinal histology appeared normal in hematoxylin and eosin stains indicates that the vascular defect is primary. In adult TSPAN12−/− mice the OPL was also not vascularized, confirming that the defect is not transient. Instead of an organized capillary bed in the IPL the TSPAN12−/− mice displayed somewhat enlarged and tortuous capillaries in the space between NFL and IPL. The thickness of inner and outer nuclear layers in TSPAN12−/− retinas was consistently reduced in adult but not developing mice, indicating that neural cells were secondarily affected by defects in vascularization.

Together, the phenotype of TSPAN12−/− mice was characterized by a largely normal development of the superficial vascular plexus and a lack of capillaries in the OPL, a phenotype that appeared to be very similar to the phenotypes reported for Fzd4 mutant mice (Xu et al. Cell 116: 883-95 (2004)) and Norrin mutant mice (Luhmann et al. Invest. Opthalmol. Vis. Sci. 46(9): 3372-82 (2005)). We therefore tested whether TSPAN12−/− mice displayed additional characteristic features that are caused by disruption of Fzd4 or its ligand Norrin. Norrin mutant mice exhibit very characteristic microaneurisms that extend from the NFL towards the inner nuclear layer at P15. Strikingly, analysis of P16 retinas of TSPAN12−/− mice by confocal microscopy revealed microaneurism-like vascular malformations that were remarkably similar to those described in the Norrin mutant mice. The similarity of these highly characteristic malformations was supported by the fact that both in Norrin mutant mice and in TSPAN12−/− mice the malformations developed at virtually identical time points. Further similarities included the aberrant expression of Meca-32 in the retinal vessels of TSPAN12−/− mice, a marker for fenestrated vessels that is normally not expressed in the retinal vasculature but is upregulated in Fzd4 mutant mice. Since Norrin and Fzd4 are a ligand/receptor pair that, in conjunction with the co-receptor LRP5, activate the canonical Wnt-pathway and promote accumulation of cytoplasmic β-catenin, our phenotypic characterization of TSPAN12−/− mice indicated that TSPAN12 may also be required for Norrin/β-catenin signaling.

### Example 2

### TSPAN12 is Involved in Wnt Signaling

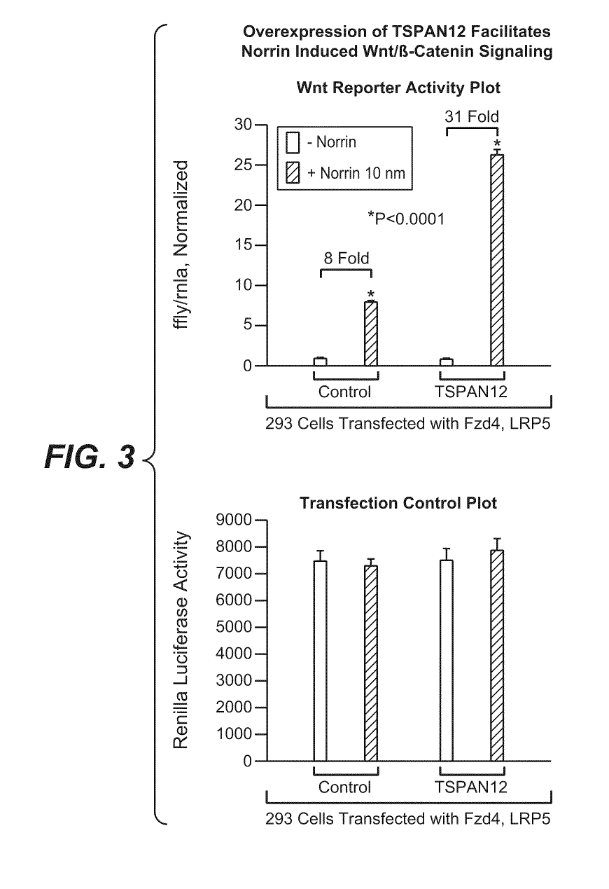

Based on the similarity between the phenotypes observed in the TSPAN12, Fzd4 and Norrin KO mice, we conducted Topflash reporter assays to determine whether TSPAN12 is involved in Norrin-induced β-catenin Wnt signaling through Fzd4. The Topflash construct consists of firefly luciferase under a promoter containing LEF/TCF consensus sites that is therefore responsive to canonical β-catenin signaling. A construct expressing renilla luciferase under a constitutive promoter is used as internal control. Cells transfected with reporter constructs and receptors were activated with 10 nM recombinant Norrin in the presence of exogenous TSPAN12 or vector control. 16-18 hours later, reporter activity (firefly activity divided by renilla activity) was determined. Norrin-mediated signaling was approximately 4-fold higher in cells overexpressing TSPAN12 than control cells (FIG. 3, left panel—right panel shows that control renilla luciferase expression was the same under all conditions). We performed similar experiments in cells where wildtype TSPAN12 mRNA was reduced using siRNA (expression was slightly below one-fifth of control levels) and found that Norrin-induced, Fzd4/LRP5-mediated expression was significantly reduced following TSPAN12 knock-down.

To further probe the specificity of the TSPAN12 effect, we conducted several experiments where we varied the frizzled constructs and/or the ligand. First we conducted experiments using cells transfected with the same vector expressing other frizzled constructs (Fzd1, Fzd2, Fzd7, Fzd10), as well as Fzd4. As shown in FIG. 4, the major effect of TSPAN12 on Norrin-mediated Wnt/β-catenin canonical signal transduction is specific to Fzd4, with a much lesser effect on Fzd10-mediated signaling. Next we analyzed the signaling in this assay using Wnt3a as the ligand to induce signaling. Here we found that TSPAN12 did not significantly enhance any FZD-mediated signaling (FIG. 5). We observed similar results using Wnt5a as the ligand.

### Example 3

### TSPAN12 is Part of the Fzd4-Receptor Complex

If TSPAN12 indeed functions during initiation of Norrin/β-catenin signaling it would be expected to colocalize and interact with components of the receptor complex. In order to test this possibility we transfected Hela cells with flag-Fzd4 (flag positioned extracellularly) and HA-TSPAN12. Plasma membrane Fzd4 was detected with a flag antibody on non-permeabilized, living cells on ice and TSPAN12 was detected subsequently after fixation and permeabilization. This staining paradigm revealed abundant Fzd4 expression on the surface of Hela cells. Fzd4 was not homogenously dispersed in the plasma membrane but instead was found to be condensed in numerous punctate areas. TSPAN12 colocalized to a large extent with Fzd4 positive punctae, and in addition occurred in intracellular structures that were not stained by the anti-flag antibody because the anti-flag staining was done on the cell surface without permeabilizing the cells. In contrast, CD9 and Fzd4 did not colocalize. When Fzd4 was substituted with Fzd5 and coexpressed with TSPAN12 we found that TSPAN12 and Fzd5 were mostly separately localized (only in rare cells that strongly expressed both proteins segregation was partially overcome).

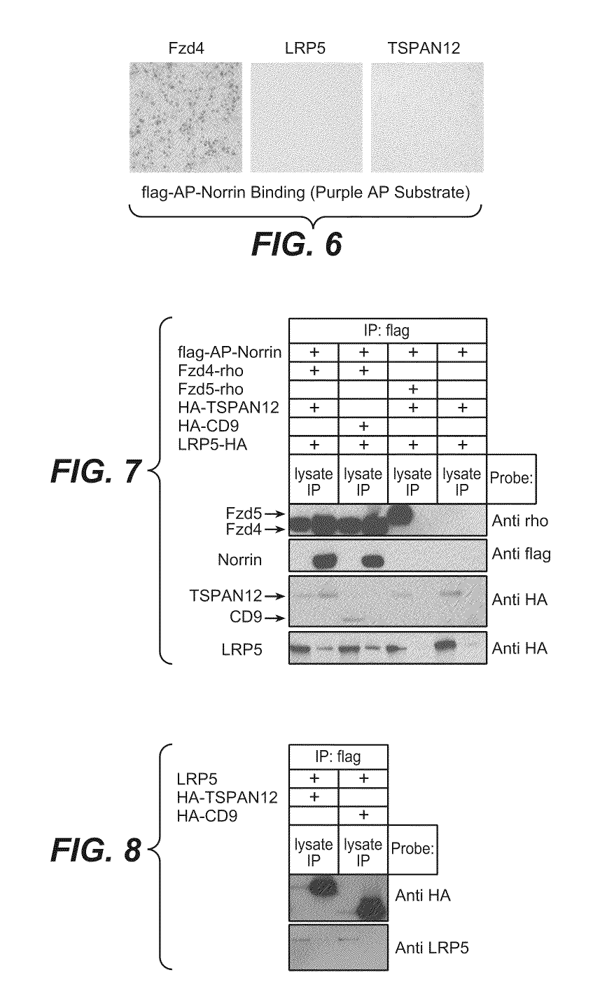

Conditioned medium containing N-terminal alkaline phosphatase fusions of Norrin (AP-Norrin) have been used to study Norrin-receptor interactions (Xu et al. supra). We transfected HeLa cells with either Fzd4, LRP5 or TSPAN12 and probed these cells with conditioned medium containing flag-AP-Norrin. Consistent with previous reports, we found that flag-AP-Norrin efficiently bound to cells expressing Fzd4 but not LRP5. Importantly, Norrin also did not bind to cells expressing TSPAN12 alone (FIG. 6). In order to probe for interactions of TSPAN12 with the receptor complex we coexpressed Fzd4, LRP5 and TSPAN12 in 293 cells and used Fzd5 as alternative Frizzled and CD9 as alternative tetraspanin in the respective controls. After incubation of these cells with conditioned medium containing flag-AP-Norrin on ice (to prevent internalization events) and extensive washing, Norrin associated membrane proteins were mildly crosslinked and then immunoprecipitated by anti-flag antibody. Norrin efficiently precipitated Fzd4 but not Fzd5. Furthermore, TSPAN12 was coprecipitated by Norrin with Fzd4 but not Fzd5, and was not precipitate when no Frizzled was present. In contrast, CD9, although expressed to a similar level as TSPAN12, was not coprecipitated with Fzd4 by Norrin (FIG. 7). Thus, TSPAN12 is physically associated with the Fzd4 receptor complex. TSPAN12 was also coprecipitated by Norrin with Fzd4 in the absence of LRP5 from detergent extracts (1% NP-40+0.1% N-dodecyl-beta-D-maltoside) when no crosslinker was used (not shown). When TSPAN12 and LRP5 were coexpressed and TSPAN12 was immunoprecipitated, no association with LRP5 was detected (FIG. 8).

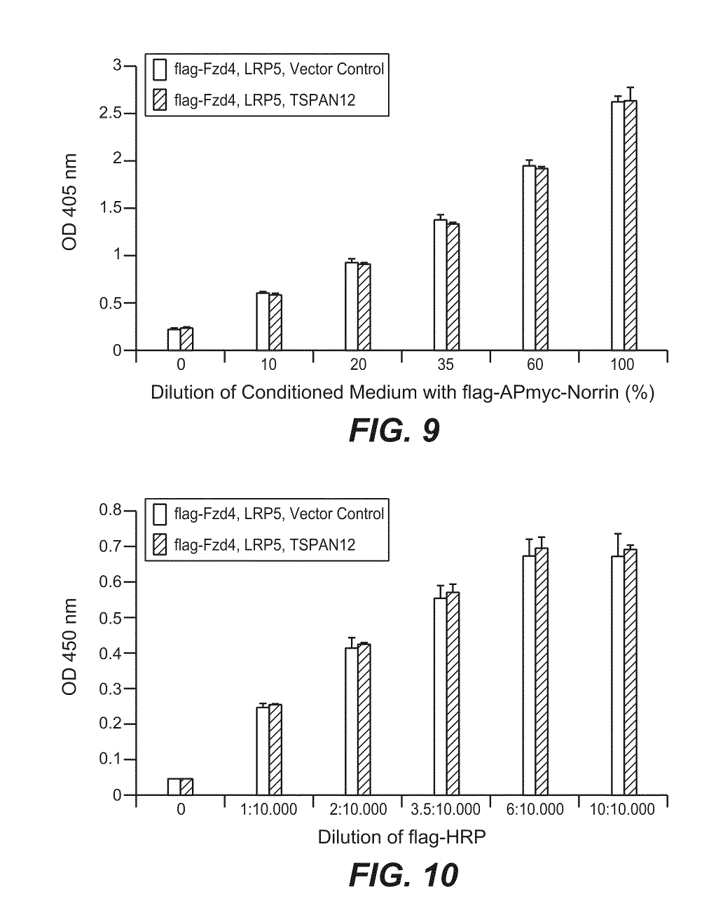

Given the strong enhancement of Norrin/β-catenin signaling by TSPAN12, we analyzed whether TSPAN12 can increase Norrin binding to Fzd4. For this purpose, Hela cells were transfected with flag-Fzd4, LRP5, TSPAN12 or vector control and subsequently probed with several dilutions of flag-AP-Norrin conditioned medium. Binding of flag-AP-Norrin to cells was similar in the presence or absence of TSPAN12 at all Norrin concentrations tested (FIG. 9). To exclude the possibility that TSPAN12 reduced Fzd4 expression levels but at the same time increased Norrin binding we directly determined the expression of Fzd4 with an HRP-coupled antibody directed against the flag peptide. Coexpression of TSPAN12 did not alter expression of Fzd4 on the plasma membrane (FIG. 10). Together, the finding that TSPAN12 does not coimmunoprecipitate with Norrin (unless Fzd4 is present) (FIG. 7) and does not enhance Norrin binding to Fzd4 (FIG. 9) indicates that TSPAN12 enhances signaling in a unique fashion. This is consistent with the functions of several other tetraspanins, which typically do not directly bind ligands but instead are thought to organize microdomains that facilitate signaling of the embedded receptors. Therefore, we next examined the possibility that TSPAN12 facilitates the interaction between components of the receptor complex.

### Example 4

### Defects of Monomeric Norrin C95R are Bypassed by TSPAN12

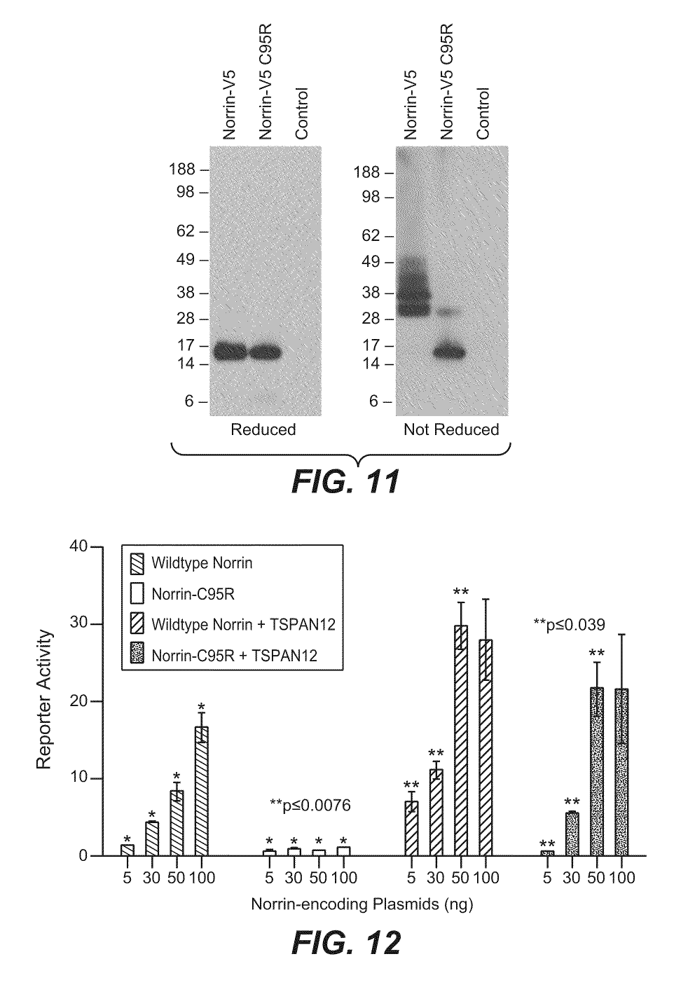

Norrin belongs to the subgroup of cysteine knot proteins that forms dimers via intermolecular disulfide bonds (Vitt et al. Mol. Endocrinol. 15(5): 681-94 (2001)) and it has been suggested that Norrin dimers can further assemble into structures of higher molecular weight. (Perez-Vilar et al. J. Biol. Chem. 272(52): 33410-15 (1997)). Norrin is strongly associated with the extracellular matrix (ECM) unless Norrin is fused to AP (Xu et al. supra). Through reduction of the intermolecular disulfide bonds Norrin can be completely converted into monomers, alternatively, mutation of the cysteine in position 95 is predicted to abolish intermolecular disulfide bonds. We expressed V5-tagged wildtype Norrin and V5-tagged Norrin C95R mutant in 293 cells, and extracted Norrin from the ECM. SDS PAGE under reducing conditions revealed monomers of wildtype and mutant Norrin that were virtually indistinguishable. Consistent with previous reports, analysis under non-reducing conditions revealed that wildtype Norrin formed dimers and assemblies of higher molecular weight. In contrast, Norrin-C95R mutant was mostly monomeric and formed no large assemblies (FIG. 11). A small fraction of total Norrin C95R formed dimers, possibly by noncovalent association or by intermolecular disulfides at a position other than C95.

We predicted that Norrin assemblies larger than monomers have the potential to bring multiple Fzd4 molecules into close proximity in the membrane and enhance Norrin/β-catenin signaling, whereas monomeric Norrin-C95R cannot. To test this idea we transfected increasing amounts of wildtype Norrin or Norrin C95R cDNA in conjunction with the receptors to induce Topflash activity in the presence or absence of TSPAN12 in 293 cells. Expression of wildtype Norrin efficiently induced Norrin/β-catenin signaling when 5-100 ng Norrin plasmid were cotransfected with FZD4 and LRP5, and the addition of TSPAN12 strongly enhanced this activity (FIG. 12). Monomeric Norrin C95R mutant, however, was virtually inactive in cells not overexpressing TSPAN12, even at the highest dose of 100 ng Norrin plasmid. Consistent with the idea that TSPAN12 can bring receptors into microdomains and allow them to situate close to each other, TSPAN12 rescued the signaling defect of Norrin C95R mutant to a large extent. Furthermore, the addition of TSPAN12 increased signaling of wildtype Norrin (FIG. 12). Together, these data indicated that Norrin multimers and TSPAN12 each provided different means to bring multiple FZD4 molecules into close proximity, and these two mechanisms act together to elicit maximal signaling.

### Example 5

### TSPAN12 Enhances Receptor Clustering

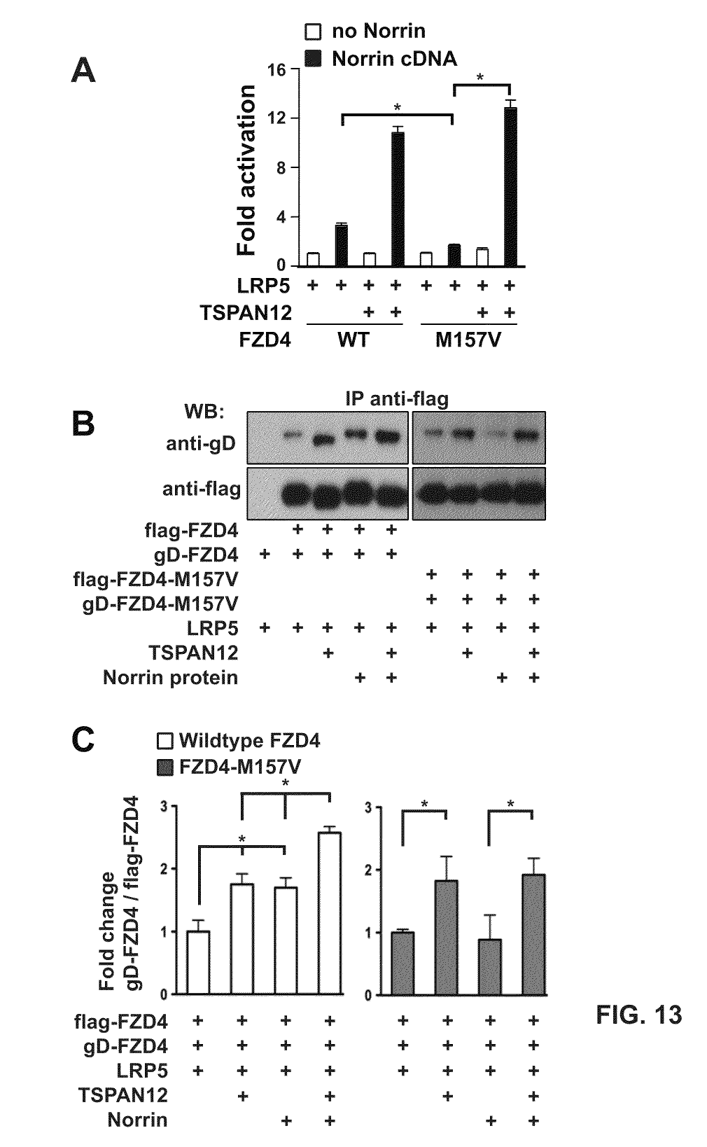

We conducted a biochemical analysis of receptor clustering using the previously described mutation FZD4-M157V, which strongly impairs Norrin/β-catenin signaling but maintains the ability to bind Norrin (Xu et al., supra). Aided by structural information (Dann et al. Nature 412:86-90 (2001)), the M157V mutation has been proposed to affect Norrin induced FZD4 dimerization and consequently multimerization (Dann et al., supra; Toomes et al., supra; Xu et al., supra). Consistent with previous reports (Xu et al., supra), we found that signaling mediated by FZD4-M157V was severely impaired. Interestingly, TSPAN12 coexpression fully rescued the signaling defect of FZD4-M157V (FIG. 13A).

We then utilized FZD4-M157V to directly investigate the role of TSPAN12 in FZD4 multimerization. 293 cells were transfected with TSPAN12 or control vector and cotransfected with FLAG™-FZD4 and gD-FZD4, or with FLAG-FZD4-M157V and gD-FZD4-M157V. Cells were incubated on ice with medium containing Norrin or no ligand. Cell lysates were immunoprecipitated with anti-FLAG antibody and probed for coimmunoprecipitation of gD-FZD4. To enable quantification of protein-protein interactions, no cross-linking reagent was used in this experiment. Similar baseline levels of association between gD-FZD4 and FLAG-FZD4 or gD-FZD4-M157V and FLAG-FZD4-M157V were detected (FIG. 13B and data not shown). Norrin and TSPAN12 each increased the amount of gD-FZD4 pulled down by FLAG-FZD4, and the combination of Norrin and TSPAN12 further increased FZD4 clustering (FIG. 13B, left panels; 13C, open bars). Importantly, the M157V mutation severely impaired the ability of Norrin to cluster gD-FZD4 with FLAG-FZD4, whereas coexpression of TSPAN12 compensated this defect (FIG. 13B, right panels; 13C, filled bars). Together, these data indicate that TSPAN12 and Norrin both promote FZD4 multimerization, and suggest that initiation of Norrin/β-catenin signaling requires i) factors that promote FZD4 multimerization and ii) activation of FZD4 by ligand binding.

We next tested whether addition of antibodies that enhance FZD4 receptor clustering could rescue the activity of FZD4-M157V. In 24-well plates, 1.6×10⁵ cells/well were transfected with a DNA mixture containing β-Catenin reporter mix (Topflash, pRL-CMV, and pCan-myc-lef-1), LRP5, and either FZD4 or FZD4-M157V. Twenty-four hours following transfection, the indicated wells received 1 μg/ml of anti-LRP5/6 antibody. One hour later, 125 ng/ml of recombinant Norrin was added to wells as indicated. Following an additional 16-hour incubation at 37° C., cells were lysed and Firefly and Renilla luciferase expression was measured using Promega Dual-Glo® Reagents. Firefly luciferase values were normalized to Renilla expression. The results are shown in Table 1. In cells expressing FZD4, reporter activity is activated by ˜6-fold in the presence of Norrin. When FZD4-M157V is expressed, Norrin activation is significantly impaired to only ˜2-fold. Adding LRP5 antibody partially rescues the signaling defect in FZD4-M157V by approximately 2-fold.

TABLE 1
Anti-LRP5/6 Antibody Partially Rescues the Defect of FZD4-M157V

| | FZD4- | | Anti- | |
|---|---|---|---|---|
| FZD4 | M157V | Norrin | LRP5/6 | Fold Activation ± St. Dev. (n = 3) |
| + | | | | 1 ± 0.10 |
| + | | + | | 6.13 ± 1.16 |
| + | | + | + | 7.51 ± 0.41 |
| | + | | | 1 ± 0.16 |
| | + | + | | 2.14 ± 0.19 |
| | + | + | + | 3.55 ± 0.11 |

### Example 6

### Generation of Anti-TSPAN12 and Anti-Norrin Antibodies

We generate anti-TSPAN12 and anti-Norrin antibodies using multiple methods. For example, we generate antibodies by immunization and hybridoma technology. We also use synthetic phage antibody libraries built on a single framework (humanized anti-ErbB2 antibody, 4D5) by introducing diversity within the complementarity-determining regions (CDRs) of heavy and light chains (Lee, et al. J. Mol. Biol. 340: 1073-93 (2004); Liang et al. J. Biol. Chem. 281: 951-61 (2006)). Plate panning with naïve libraries is performed against His-tagged human TSPAN12 immobilized on MaxiSorp™ immunoplates. After four rounds of enrichment, clones are randomly picked and specific binders are identified using phage ELISA. The resulting hTSPAN12 binding clones are further screened with His-tagged murine TSPAN12 protein to identify cross-species clones. For each positive phage clone, variable regions of heavy and light chains are subcloned into pRK expression vectors that are engineered to express full-length IgG chains. Heavy chain and light chain constructs are co-transfected into 293 or CHO cells, and the expressed antibodies are purified from serum-free medium using protein A affinity column. Purified antibodies are tested by ELISA for binding to recombinant TSPAN12 or Norrin, and by FACS for binding to stable cell lines expressing either full-length human TSPAN12 or murine TSPAN12 in conjunction with FZD4 or expressing human or murine Norrin. The antibodies are then tested for blocking the enhancement of Norrin-mediated, FZD4/LRP5-mediated Wnt reporter activity by TSPAN12 (anti-TSPAN12 antibodies) or to block Norrin-induced signaling (anti-Norrin antibodies). For affinity maturation, phage libraries with three different combination of CDR loops (CDR-L3, -H1, and -H2) derived from the initial clone of interest are constructed by soft randomization strategy so that each selected position is mutated to a non-wild type residue or maintained as wild type at about 50:50 frequency (Liang et al., 2006, above). High affinity clones are then identified through four rounds of solution phase panning against both human and murine His-tagged TSPAN12 proteins with progressively increased stringency.

### Example 7

### Murine Models of Ocular Disease

We test the antibodies or TSPAN12 polypeptides in murine models. For the murine ROP model, pups are placed in 75% oxygen (hyperoxia) for a period of five days beginning at P7. At P12, the animals are returned to room air condition (normoxia) and maintained for another five days (P17). Anti-TSPAN12, anti-Norrin antibody or TSPAN12 large extracellular loop (e.g. Ho et al. supra) is injected intravitreally into the P12 animals. Multiple dose levels and frequencies are done based on predictions determined by antagonist affinity and stability. On P17, the right-eye is placed in 4% PFA and the left-eye was placed in Davidson's fixative. The cornea and lens are removed from left eyes for paraffin processing and sectioning. Eye cups are placed in the block with the iris side down, facing the sectioning surface. Sections spaced 16 microns apart are obtained and analyzed for neovascularization using the Aperio ScanScope® equipped with a custom designed nuclear algorithm. Neovascular tufts intimately associated with the NFL are identified as regions of interest for the algorithm quantification. A minimum of 30 sections per case, containing neovascular nuclei are quantified for assessment of retinal neovascularization.

We also test the antibodies and polypeptides in a murine laser-induced choroidal neovascularization model.

The foregoing written specification is considered to be sufficient to enable one skilled in the art to practice the invention. However, various modifications of the invention in addition to those shown and described herein will be apparent to those skilled in the art from the foregoing description and fall within the scope of the appended claims.

What is claimed is:

1. A method of reducing or inhibiting angiogenesis in a subject having an ocular disease or condition associated with angiogenesis, comprising administering to the subject a TSPAN12 antagonist.

2. The method of claim 1, wherein the TSPAN12 antagonist is an anti-TSPAN12 antibody.

3. The method of claim 1, wherein the TSPAN12 antagonist comprises a polypeptide fragment of TSPAN12.

4. The method of claim 3, wherein the polypeptide fragment of TSPAN12 comprises an extracellular domain of TSPAN12.

5. The method of claim 3 or 4, wherein the TSPAN12 antagonist further comprises an immunoglobulin constant region.

6. The method of claim 5, wherein the immunoglobulin constant region is an IgG Fc.

7. The method of claim 1, wherein the ocular disease or condition is selected from the group consisting of: diabetic retinopathy, choroidal neovascularization (CNV), age-related macular degeneration (AMD), diabetic macular edema (DME), pathological myopia, von Hippel-Lindau disease, histoplasmosis of the eye, central retinal vein occlusion (CRVO), branched central retinal vein occlusion (BRVO), corneal neovascularization, retinal neovascularization, retinopathy of prematurity (ROP), subconjunctival hemorrhage, and hypertensive retinopathy.

8. The method of claim 7, wherein the ocular disease or condition is selected from the group consisting of diabetic retinopathy, AMD, DME, CRVO, and BRVO.

9. The method of claim 1, further comprising administering to the subject a second anti-angiogenic agent.

10. The method of claim 9, wherein the second anti-angiogenic agent is administered prior to or subsequent to the administration of the TSPAN12 antagonist.

11. The method of claim 9, wherein the second anti-angiogenic agent is administered concurrently with the TSPAN12 antagonist.

12. The method of claim 9, wherein the second anti-angiogenic agent is an antagonist of Norrin or vascular endothelial cell growth factor (VEGF).

13. The method of claim 12, wherein the Norrin antagonist is an anti-Norrin antibody.

14. The method of claim 12, wherein the VEGF antagonist is an anti-VEGF antibody.

15. The method of claim 14, wherein the anti-VEGF antibody is ranibizumab.

16. A method of reducing or inhibiting angiogenesis in a subject having an ocular disease or condition associated with angiogenesis, comprising administering to the subject a Norrin antagonist.

17. The method of claim 16, wherein the Norrin antagonist is an anti-Norrin antibody.

18. The method of claim 16, wherein the ocular disease or condition is selected from the group consisting of: diabetic retinopathy, CNV, AMD, DME, pathological myopia, von Hippel-Lindau disease, histoplasmosis of the eye, CRVO, BRVO, corneal neovascularization, retinal neovascularization, ROP, subconjunctival hemorrhage, and hypertensive retinopathy.

19. The method of claim 18, wherein the ocular disease is selected from the group consisting of diabetic retinopathy, AMD, DME, CRVO, and BRVO.

20. The method of claim 16, further comprising administering to the subject a second anti-angiogenic agent.

21. The method of claim 20, wherein the second anti-angiogenic agent is administered prior to or subsequent to the administration of the Norrin antagonist.

22. The method of claim 20, wherein the second anti-angiogenic agent is administered concurrently with the Norrin antagonist.

23. The method of claim 20, wherein the second anti-angiogenic agent is an antagonist of VEGF.

24. The method of claim 23, wherein the VEGF antagonist is an anti-VEGF antibody.

25. The method of claim 23, wherein the anti-VEGF antibody is ranibizumab.

26. A method of treating an ocular disease or condition associated with undesired angiogenesis in a subject comprising administering to the subject a TSPAN12 antagonist.

27. The method of claim 26, wherein the TSPAN12 antagonist is an anti-TSPAN12 antibody.

28. The method of claim 26, wherein the TSPAN12 antagonist comprises a polypeptide fragment of TSPAN12.

29. The method of claim 27, wherein the polypeptide fragment of TSPAN12 comprises an extracellular domain of TSPAN12.

30. The method of claim 28 or 29, wherein the TSPAN12 antagonist further comprises an immunoglobulin constant region.

31. The method of claim 30, wherein the immunoglobulin constant region is an IgG Fc.

32. The method of claim 26, wherein the ocular disease or condition is selected from the group consisting of: proliferative retinopathies including proliferative diabetic retinopathy, CNV, AMD, diabetic and other ischemia-related retinopathies, DME, pathological myopia, von Hippel-Lindau disease, histoplasmosis of the eye, CRVO, BRVO, corneal neovascularization, retinal neovascularization, ROP, subconjunctival hemorrhage, and hypertensive retinopathy.

33. The method of claim 32, wherein the ocular disease or condition is selected from the group consisting of diabetic retinopathy, AMD, DME, CRVO, and BRVO.

34. The method of claim 26, further comprising administering to the subject a second anti-angiogenic agent.

35. The method of claim 34, wherein the second anti-angiogenic agent is administered prior to or subsequent to the administration of the TSPAN12 antagonist.

36. The method of claim 34, wherein the second anti-angiogenic agent is administered concurrently with the TSPAN12 antagonist.

37. The method of claim 34, wherein the second anti-angiogenic agent is an antagonist of Norrin or VEGF.

38. The method of claim 37, wherein the Norrin antagonist is an anti-Norrin antibody.

39. The method of claim 37, wherein the VEGF antagonist is an anti-VEGF antibody.

40. The method of claim 39, wherein the anti-VEGF antibody is ranibizumab.

41. A method of treating an ocular disease or condition associated with undesired angiogenesis in a subject comprising administering administering to the subject a Norrin antagonist.

42. The method of claim 41, wherein the Norrin antagonist is an anti-Norrin antibody.

43. The method of claim 41, wherein the ocular disease or condition is selected from the group consisting of: proliferative retinopathies including proliferative diabetic retinopathy, CNV, AMD, diabetic and other ischemia-related retinopathies, DME, pathological myopia, von Hippel-Lindau disease, histoplasmosis of the eye, CRVO, BRVO, corneal neovascularization, retinal neovascularization, ROP, subconjunctival hemorrhage, and hypertensive retinopathy.

44. The method of claim 43, wherein the ocular disease is selected from the group consisting of diabetic retinopathy, AMD, DME, CRVO, and BRVO.

45. The method of claim 41, further comprising administering to the subject a second anti-angiogenic agent.

46. The method of claim 45, wherein the second anti-angiogenic agent is administered prior to or subsequent to the administration of the Norrin antagonist.

47. The method of claim 45, wherein the second anti-angiogenic agent is administered concurrently with the Norrin antagonist.

48. The method of claim 45, wherein the second anti-angiogenic agent is an antagonist of VEGF.

49. The method of claim 48, wherein the VEGF antagonist is an anti-VEGF antibody.

50. The method of claim 49, wherein the anti-VEGF antibody is ranibizumab.
